(19) United States
(12) Patent Application Publication
(10) Pub. No.: US 2003/0186291 A1
(43) Pub. Date: Oct. 2, 2003

(54) Genetically engineered phiC31-integrase genes

(76) Inventors: Nicole Faust, Roesrath (DE); Susanne Andreas, Neuss (DE)

Correspondence Address: JAN P. BRUNELLE, EXELIXIS, INC., 170 HARBOR WAY, P.O. BOX 511, SOUTH SAN FRANCISCO, CA, 94083-0511, US

(21) Appl. No.: 10/359,050

(22) Filed: Feb. 5, 2003

Related U.S. Application Data
(60) Provisional application No. 60/354,741, filed on Feb. 6, 2002.

(51) Int. Cl. C12Q 1/68; C07H 21/04; C12N 9/22; C12P 21/02; C12N 5/06

(52) U.S. Cl. 435/6; 435/69.1; 435/199; 435/320.1; 435/325; 536/23.2

(57) ABSTRACT

Genetically engineered nucleic acid molecules encoding phiC31-integrase are described. These nucleic acid molecules, referred to as C31-Int genes, comprise sequences optimized for expression in eukaryotic host cells. Vectors, microorganisms, vertebrate cells, and transgenic organisms comprising optimized C31-Int genes are also described. PhiC31 integrase proteins encoded by the optimized C31-Int genes, as well as methods of recombining a DNA molecules containing phiC31 integrase recognition sequences are provided.

### REFERENCE TO RELATED APPLICATIONS

This application claims priority to U.S. provisional patent application No. 60/354,741 filed Feb. 6, 2002. The contents of the prior application is hereby incorporated in its entirety.

### BACKGROUND OF THE INVENTION

The ability to generate controlled and permanent modifications to genomes of eukaryotic cells and organisms has important research applications, including the study of gene function, the creation of disease models, medical applications such as gene therapy, and the design of economically important animals and crops.

Site-specific recombinases (SSRs), such as the bacteriophage P1-derived Cre recombinase, provide important tools for engineering eukaryotic genomes. SSRs recognize specific DNA sequences (“recognition sites” or “recognition sequences”) and catalyze recombination between two recognition sites. Cre recombinase, for example, recognizes the 34 base pair (bp) loxP motif (Austin et al., Cell 25, 729-736 (1981)). If the two sites are located on the same DNA molecule in the same orientation, the intervening DNA sequence is excised by the recombinase from the parental molecule as a closed circle, leaving one recognition site on each of the reaction products. If the two sites are in inverted orientation, the recognition-site flanked region is inverted through recombinase-mediated recombination. Alternatively, if the two recognition sites are located on different molecules, recombinase-mediated recombination will lead to integration of a circular molecule or translocation between two linear molecules. These features make SSRs extremely useful for a number of applications in mammalian systems, including conditional activation of transgenes in mice, chromosome engineering to obtain deletions, translocations or inversions, removal of selection marker genes, gene replacement, targeted insertion of transgenes, and the activation or inactivation of genes by inversion (Nagy, Genesis, 26, 99-109 (2000); Cohen-Tannoudji et al., Mol. Hum. Reprod. 4, 929-938 (1998)).

In addition to Cre, a few recombinases have been shown to exhibit some activity in mammalian cells. The best characterized examples are the yeast-derived FLP and Kw recombinases, which exhibit optimal activity at 30° C. and are unstable at 37° C. (Buchholz et al., Nature Biotech., 16, 657-662 (1998); Ringrose et al., Eur. J. Biochem., 248, 903-912). Other recombinases that show some activity in mammalian cells include a mutant integrase of phage lamda, the integrases of phage HK022, mutant gamma delta-resolvase and beta-recombinase (Lorbach et al., J. Mol. Biol., 296, 1175-81 (2000); Kolot et al., Mol. Biol. Rep. 26, 207-213 (1999); Schwikardi et al., FEBS Lett., 471, 147-150 (2000); Diaz et al., J. Biol. Chem., 274, 6634-6640 (1999)). The integrase of phage phiC31 (C31-Int) has been found to work in mammalian cells (EP00124629.7; U.S.60/311,876; Groth et al., Proc. Natl. Acad. Science, 97, 5995-6000 (2000)). Moreover, an improved version of the phiC31 integrase has been developed. This modified C31-Int (C31-Int(CNLS)) carries a C-terminal nuclear localization signal (NLS) and displays a recombination efficiency in mammalian cells-that is significantly enhanced over the wild type form and is comparable to that of Cre recombinase (EP00124629.7; U.S.60/311,876). This makes the C31-Int a valuable tool for mammalian genome modification.

Unlike FLP or Kw, and like the majority of SSRs, the phage derived C31-Int is normally expressed in a prokaryotic organism. Examples of other phage integrase systems include coliphage P4 recombinase, Listeria phage recombinase, bacteriophage R4 Sre recombinase, CisA recombinase, XisF recombinase and transposon Tn4451 TnpX recombinase (Stark et al. Trends in Genetics 8, 432-439 (1992); Hatfull & Gridley, in Genetic Recombination. Eds. Kucherlipati & Smith, Am. Soc. Microbiol., Washington D.C., 357-396 (1988)).

For use in eukaryotic systems, SSRs should be expressed at high levels. However, expression of prokaryotic genes in eukaryotic systems can face several problems:

The first problem is codon usage. Through the redundancy of the genetic code, most amino acids are encoded by multiple codons. It has been observed that the codon for a given amino acid is not randomly chosen. Rather, certain codons are preferred, and the frequency of usage of particular codons varies by organism (Ikemura, Mol. Biol Evol. 2, 13-34 (1985); Zhang et al., Gene 105, 61-72 (1991)). The relative frequency of codons is usually correlated to the abundance of the corresponding tRNA (Duret, Trends Genet. 16, 287-289 (2000); Moriyama and Powell, J. Mol. Evol. 45, 514-523 (1997)). For many organisms it has been found that highly expressed genes show a bias in codon usage: they show an over-proportional usage of codons for abundant tRNAs (Grantham et al., Nucleic Acids Res. 9, r43-r74 (1981)). Prokaryotic genes may therefore have a codon composition that is not favorable for high-level expression in eukaryotic systems.

The second potential problem is splicing. The splicing process is unique to eukaryotic cells and does not occur in prokaryotes. For this reason prokaryotic genes may contain sequence motifs that are recognized as splice donors or splice acceptors when the gene is integrated into the genome of eukaryotic cells. This can lead to aberrant and undesired splicing of the prokaryotic transgene, resulting in a truncated gene product.

The third potential problem is methylation of the DNA dinucleotide motif CpG in vertebrate cells. Methylcytosine can undergo spontaneous deamination to thymine, resulting in a C to T transition in the DNA sequence. For this reason the CpG dinucleotide is statistically underrepresented in the vertebrate genome. On the other hand, in mammals DNA methylation is often associated with gene silencing (Chomet, Curr. Opin. Cell Biol. 3, 438-443 (1991); Razin, EMBO J. 17, 4905-4908 (1998)). CpG rich prokaryotic genes are therefore prone to gene silencing if introduced into mammalian organisms (Cui et al., Transgenic Res. 3, 182-194 (1994)). Further, unmethylated CpG dinucleotides that are flanked by two 5′ purines and two 3′ pyrimidines (the “immuno-stimulatory CpG motif”) have been shown to stimulate an immune response in vivo and in vitro (Krieg et al., 1995, Nature 374:546-9; Sato et al., Science 273, 352-354 (1996)).

All these differences between prokaryotic and eukaryotic (mammalian in particular) gene architecture can hamper efficient expression of a phage or bacteria-derived site-specific recombinase in a mammalian organism such as the mouse. For Cre codon-optimized genes with improved expression in mammals have been described (PCT EP01 07729; Koresawa et al., J. Biochem. 127, 367-372 (2000)). However, codon-optimisation has to be performed individually for each new gene, taking into account all factors that can influence gene expression.

### SUMMARY

The present invention provides genetically engineered nucleic acid molecules encoding phiC31-integrase. These nucleic acid molecules, referred to as C31-Int genes, comprise sequences optimized for expression in eukaryotic host cells. In a preferred embodiment, a C31-Int gene comprises at least 306, 430, or 550 codons that are optimal for expression in the host cell. Preferred host cells are from mouse, rat, human, rabbit, and teleost.

In some embodiments, the optimized C31-Int gene has been further engineered to remove sequences matching consensus 5′ splice donor sequences or consensus 3′ splice acceptor sequences, and/or CpG dinucleotides. The C31-Int gene may contain fewer than 200, 150, 100, or 50 CG dinucleotides, and/or contain few or no immuno-stimulatory CpG motifs with the sequence RRCGYY. A C31-Int gene of the present invention may further comprise a Kozak consensus sequence at the translational start codon, a second termination codon positioned 3′ to the first translational termination codon, and/or a nucleotide sequence encoding a 3′ nuclear localization signal.

The invention further provides vectors, microorganisms, vertebrate cells, and transgenic organisms comprising optimized C31-Int genes. In some embodiments, a vertebrate cell comprising a C31-Int gene further comprises phiC31 integrase recognition sequences. The invention provides phiC31 integrase proteins encoded by the optimized C31-Int genes, as well as methods of recombining a DNA molecules containing phiC31 integrase recognition sequences, comprising contacting the DNA molecule with a phiC31 integrase encoded by a C31-Int gene of the invention.

### BRIEF DESCRIPTION OF THE FIGURES

FIG. 1 depicts the ROSA26 targeting vector for C31-Int (CNLS) and C31-Int (CNLS)-CO.

FIG. 2 depicts ROSA26 locus of the C31 reporter mice carrying a C31 substrate reporter construct.

### DETAILED DESCRIPTION

The invention provides nucleic acid sequences encoding the recombinase phiC31-Integrase (“C31-Int”), where the nucleic acid sequences have been genetically engineered for expression in a eukaryotic host. The term “native (or wild-type) C31-Int gene” refers to a gene that is naturally occurring and/or has not been modified through human intervention, as presented in SEQ ID NO:1. The protein sequence encoded by the native C31-Int is provided in SEQ ID NO:2. The changes introduced into the coding sequence are typically “silent mutations,” meaning that they do not result in changes to the amino acid sequence. As used herein, the term “C31-Int gene” refers to the nucleic acid molecule encoding a C31-Int protein. The C31-Int gene typically includes a translational initiation codon, as well as a translational termination codon. Gene regulatory sequences, including upstream enhancers and/or promoters and a downstream polyadenylation signal, all of which may be heterologous, are usually operably linked to the C31-Int gene. The coding sequence may also comprise heterologous, in-frame coding sequences fused to the recombinase coding sequence. As used herein, the term “optimized C31-Int gene” refers to a C31-Int gene that has been genetically engineered to comprise at least one of the modifications disclosed herein. The invention further provides methods of optimizing the C31-Int coding sequence.

The present invention is described using the following conventions for describing nucleic acid sequences. Sequences are presented in the 5′ to 3′ direction. Nucleotides may be referred to by the bases they comprise. “A” represents a nucleotide comprising the purine base adenine; “G” represents a nucleotide comprising the purine base guanine; “C” represents a nucleotide comprising the pyrimidine base cytosine, and “T” represents a nucleotide comprising the pyrimidine base thymine. Thus when it is said that, for instance, the fourth and fifth position of a nucleotide sequence consist of a G and a T, it is meant that these positions in the corresponding nucleic acid molecule consist of a nucleotide comprising a guanine base and a nucleotide comprising a thymine base, respectively. “Y” represents either a T or a C; “R” represents either an A or a G, and “N” represents any base (A, C, G, or T). With respect to sequences that represent splice junctions, capital letters represent exon sequence, and lower case letters represent intron sequence. Letters that are within brackets represent the alternative bases that can occur at the given position; percentage values may be included within brackets to indicate the frequencies at which particular bases occur. Intron and exon sequences are depicted in lower and upper case letters for convenience only. It will be understood that with respect to a nucleic acid molecule, there is no structural difference between nucleotides designated as intron or exon sequence. Moreover, in terms of the phiC31 gene sequences of the present invention, all bases will be coding sequence (i.e., “exon”) with respect to the integrase (i.e., even when they are may be recognized as splice junctions and are therefore depicted using include some lower case letters).

In a preferred embodiment, the C31 nucleic acid sequence is “codon optimized.” To optimize the C31-Int gene sequence for expression in eukaryotic species, silent mutations are introduced into the coding sequence to change the codon encoding a given amino acid to the codon that is most frequently used in the respective host. Such codon usage data is available for a large number of eukaryotic organisms, and, as sequencing of eukaryotic genomes and expressed sequences progresses, is continually being generated (see for instance, the website at www.kazusa.or.jp/codon/). Table 1 contains data for mouse (Mus musculus), rat (Rattus norvegicus), rabbit (Oryctolagus cuniculus), human (Homo sapiens), and zebrafish (Danio rerio). Frequencies of codon usage per thousand are shown for each triplet. The data source was the codon usage database (website at www.kazusa.or.jp/codon/).

TABLE 1
Codon frequencies for bacteriophage phiC31, mouse, rat, rabbit, zebrafish,
and human.

| frequency per thousand | | | | | | | frequency per thousand | | | | | | |
|---|---|---|---|---|---|---|---|---|---|---|---|---|---|
| | | mus | rattus | oryctolagus | homo | denio | | | mus | rattus | oryctolagus | homo | denio |
| triplet | phiC31 | musculus | norvegicus | cuniculus | sapiens | rerio | triplet | phiC31 | musculus | norvegious | cuniculus | sapiens | rerio |
| UUU | 2.7 | 16.0 | 16.3 | 16.7 | 17.0 | 16.9 | CUU | 24.3 | 12.3 | 12.0 | 10.0 | 12.8 | 11.6 |
| UUC | 27.4 | 21.6 | 24.2 | 29.0 | 20.5 | 21.8 | CUC | 14.9 | 19.6 | 20.7 | 23.4 | 19.3 | 16.8 |
| UUA | 0.0 | 6.0 | 5.4 | 5.2 | 7.3 | 6.1 | CUA | 2.2 | 7.5 | 7.2 | 4.9 | 7.0 | 5.7 |
| UUG | 6.1 | 12.5 | 12.2 | 10.8 | 12.5 | 11.6 | CUG | 27.2 | 39.3 | 41.5 | 48.2 | 39.7 | 35.3 |
| UCU | 3.7 | 15.4 | 14.4 | 10.4 | 14.8 | 16.8 | CCU | 5.2 | 18.7 | 17.1 | 12.8 | 17.3 | 16.7 |
| UCC | 12.5 | 18.1 | 18.2 | 19.4 | 17.5 | 18.4 | CCC | 1.2 | 19.1 | 18.4 | 20.7 | 20.0 | 15.5 |
| UCA | 2.6 | 11.2 | 10.6 | 7.7 | 11.9 | 13.2 | CCA | 1.0 | 17.3 | 15.7 | 11.9 | 16.7 | 16.1 |
| UCG | 19.4 | 4.5 | 4.4 | 5.4 | 4.5 | 7.2 | CCG | 32.4 | 6.8 | 6.5 | 8.3 | 7.0 | 10.6 |
| UAU | 5.7 | 11.8 | 11.5 | 9.9 | 12.1 | 12.8 | CAU | 2.8 | 9.9 | 9.1 | 7.4 | 10.5 | 10.5 |
| UAC | 21.5 | 16.8 | 17.7 | 19.9 | 15.8 | 19.5 | CAC | 15.0 | 15.3 | 14.8 | 16.3 | 14.9 | 17.4 |
| UAA | 0.7 | 0.6 | 0.6 | 0.5 | 0.7 | 0.8 | CAA | 3.1 | 11.6 | 10.6 | 9.1 | 12.0 | 11.7 |
| UAG | 0.8 | 0.5 | 0.5 | 0.5 | 0.5 | 0.4 | CAG | 20.4 | 34.5 | 33.0 | 32.6 | 34.5 | 33.5 |
| UGU | 2.4 | 10.9 | 9.6 | 8.2 | 10.0 | 11.1 | CCU | 12.8 | 4.7 | 4.9 | 3.8 | 4.6 | 6.9 |
| UGC | 8.2 | 12.6 | 12.1 | 13.2 | 12.3 | 13.6 | CGC | 29.2 | 10.1 | 10.2 | 13.1 | 10.8 | 10.5 |
| UGA | 2.7 | 1.1 | 1.0 | 1.0 | 1.3 | 1.1 | CGA | 5.0 | 6.7 | 6.6 | 5.1 | 6.3 | 6.9 |
| UGG | 19.3 | 12.9 | 13.2 | 14.0 | 12.9 | 11.8 | CGG | 21.3 | 10.5 | 10.7 | 11.5 | 11.6 | 7.5 |
| AUU | 14.1 | 14.6 | 15.4 | 14.5 | 15.8 | 15.9 | GUU | 17.6 | 10.1 | 10.0 | 9.0 | 10.9 | 12.1 |
| AUC | 19.5 | 22.9 | 26.1 | 30.2 | 21.6 | 24.2 | GUC | 29.9 | 15.6 | 16.9 | 18.1 | 14.6 | 14.5 |
| AUA | 0.9 | 6.6 | 6.6 | 6.0 | 7.2 | 6.8 | GUA | 3.8 | 6.9 | 7.0 | 4.9 | 7.0 | 6.1 |
| AUG | 18.6 | 22.2 | 23.7 | 24.4 | 22.3 | 27.0 | GUG | 29.4 | 29.0 | 30.7 | 33.3 | 28.8 | 26.5 |
| ACU | 8.0 | 13.2 | 12.8 | 10.1 | 12.9 | 13.6 | GCU | 18.7 | 19.7 | 19.5 | 15.7 | 18.5 | 19.3 |
| ACC | 18.5 | 19.6 | 20.6 | 22.1 | 19.3 | 18.7 | GCC | 49.2 | 26.7 | 28.1 | 33.9 | 28.3 | 20.9 |
| ACA | 2.0 | 15.6 | 15.1 | 11.6 | 14.9 | 15.6 | GCA | 9.5 | 15.5 | 15.2 | 12.8 | 15.9 | 15.5 |
| ACG | 39.5 | 6.1 | 6.4 | 8.9 | 6.3 | 8.4 | GCG | 45.6 | 7.1 | 6.9 | 8.8 | 7.5 | 9.7 |
| AAU | 3.5 | 15.5 | 15.0 | 13.3 | 17.0 | 15.7 | GUN | 6.6 | 21.4 | 20.7 | 17.9 | 22.4 | 21.3 |
| AAC | 24.7 | 21.5 | 22.6 | 24.2 | 19.8 | 26.8 | GAC | 63.1 | 27.5 | 28.5 | 30.9 | 26.1 | 28.8 |
| AAA | 1.3 | 21.3 | 20.5 | 20.3 | 24.0 | 25.2 | GAA | 26.7 | 26.9 | 25.9 | 24.7 | 29.1 | 21.1 |
| AAG | 45.5 | 34.6 | 35.3 | 35.2 | 32.6 | 28.6 | GAG | 34.9 | 40.3 | 40.8 | 43.7 | 40.2 | 38.8 |
| AGU | 3.5 | 12.2 | 11.4 | 8.5 | 12.0 | 13.0 | GGU | 17.1 | 11.7 | 11.2 | 9.0 | 10.8 | 14.0 |
| AGC | 11.6 | 20.0 | 19.3 | 19.1 | 19.3 | 21.6 | GGU | 44.5 | 22.9 | 22.6 | 26.5 | 22.7 | 19.1 |
| AGA | 0.7 | 11.4 | 10.4 | 9.2 | 11.5 | 12.7 | GGA | 7.0 | 17.3 | 16.4 | 14.9 | 16.4 | 21.1 |
| AGG | 1.3 | 11.6 | 11.4 | 10.4 | 11.3 | 10.3 | GGG | 17.5 | 15.9 | 15.8 | 17.0 | 16.4 | 10.2 |

As used herein, the terms “codon that is optimal for expression in the eukaryotic host cell” and “optimal host codon” refer to the codon sequence that is most utilized by the particular host. If two codon sequences are essentially equally utilized (e.g., within approximately 1-2%), the optimal codon can refer to either of these sequences. Table 1 (as well as Table 4, below) further provides the second, third, fourth, fifth and sixth most prevalent codon sequences for the particular species. It will be understood that different amino acids are encoded 1, 2, 3, 4, or 6 codons (e.g., Met is encoded by one codon, Cys by two, and Ser by six). Thus, general reference to a second, third, fourth, etc. most prevalent codon refers to as many codons exist for any particular amino acid. For a sequence optimized for a particular host, preferably at least 50%, more preferably at least 70%, and most preferably at least 90% of the codons in the codon-optimized gene will be identical to an optimal host codon. The nucleotide sequence encoding C31-Int preferably comprises at least 306, more preferably at least 430, and more preferably at least 550 codons that are optimal for expression in the particular host.

The sequence may be further engineered to eliminate potential splice sites that can lead to aberrant splicing after integration into the host genome. The codon-optimized sequence is analyzed for motifs matching either the splice donor or the splice acceptor consensus sequences. The nine nucleotide consensus for the 5′ splice donor site is characterized by the sequence [A,C]Aggt[a,g]agt (Zhuang and Weiner, Cell 46, 827-835 (1986); Stamm et al., DNA and Cell Biology, 19, 739-756 (2000)). Only the GT dinucleotide at the exon/intron boundary (i.e., the G and T in, respectively, the fourth and fifth positions of the consensus) is 100% conserved. At the other positions, alternative nucleotide usage is found with certain frequencies. The consensus sequence is therefore more appropriately described by [C40%, A30%, G20%, C10%] [A70%, G10%, C10%, T10%] [G70%, A15%, T10%, C5%] [g100%] [t100%] [a60%, g30%, c5%, t5%] [a55%, t15%, c12.5%, g12.5%] [g70%, a12.5%, t10%, c7.5%] [t50%, a20%, g20%, c10%]. Sequences matching variations of the consensus may also be changed to sequences less favorable for splicing through silent mutations. Such silent mutations most preferably replace the optimal codon with the second most prevalent codon and may also replace the optimal codon with the third or fourth most prevalent codon.

In one embodiment, the nucleic acid molecule of the present invention does not contain a splice donor sequence, wherein the splice donor sequence is AAGgtaagt, AAGgtgagt, CAGgtaagt, or CAGgtgagt. In other embodiments, the nucleic acid molecule of the present invention does not contain a splice donor sequence comprising nine contiguous nucleotides, wherein the fourth and fifth are, respectively, G and T, and wherein at least three, four, or five of the nucleotides in the first, second, third, six, seventh, eighth, and/or ninth positions are identical to the nucleotide in the corresponding position in the sequence AAGgtaagt, AAGgtgagt, CAGgtaagt, or CAGgtgagt. In each case, the “corresponding nucleotide” is determined simply by counting, starting with “1” for the first nucleotide of a 9-nucleotide sequence. As illustration, a nucleic acid molecule that comprises the sequence “CTCGTCATT” would be said to contain a splice donor sequence where the fourth and fifth positions are G and T, respectively, and three additional bases—those in the first, seventh, and ninth positions—are identical to the nucleotide in the corresponding position of CAGgtaagt.

The nucleic acid of the present invention may alternatively or additionally be engineered to eliminate potential 3′ splice acceptor sequences. The 3′ splice acceptor site is characterized by the twelve base consensus sequence yyyyyyyncagG (Moore, Nature Struct. Biol. 7, 14-16 (2000); Stamm et al., DNA and Cell Biology, 19, 739-756 (2000)). However, only the AG dinucleotide at the intron/exon boundary (i.e., the A and G in, respectively, the tenth and eleventh positions of the consensus) is 100% conserved. At the other positions, alternative nucleotide usage is found with certain frequencies. The consensus sequence is therefore more appropriately described by yyyyyyyn [c80%, t20%] [a100%] [g100%] [G50%, A20%, C20%, T10%] (Stamm et al., DNA and Cell Biology, 19:739-756, 2000). Sequences matching any of these variations of the consensus sequence may be changed to sequences less favorable for splicing through silent mutations. Such silent mutations most preferably replace the optimal codon with the second most prevalent codon and may also replace the optimal codon with the third or fourth most prevalent codon.

In one embodiment, the nucleic acid molecule of the present invention does not contain a splice acceptor sequence, wherein the splice acceptor sequence is yyyyyyyncagG (SEQ ID NO:3) or yyyyyyyntagG (SEQ ID NO:4). It will be understood that since each “Y” can represent either C or T, and “N” can represent any base, each sequence of SEQ ID NO:3 and each sequence of SEQ ID NO:4 actually represents 512 (2⁷×4) distinct sequences. In other embodiments, the nucleic acid molecule of the present invention does not contain a splice acceptor sequence comprising twelve contiguous bases, wherein the ninth position is a C or T, wherein the tenth and eleventh bases are, respectively, A and G, and wherein at least four or five of the bases in the first, second, third, fourth, fifth sixth, seventh, and twelfth positions are identical to the base in the corresponding position in any of the sequences of SEQ ID NO:3 or 4. For illustration, a nucleic acid molecule that comprises the sequence “CTACAAGGTAGG” would be said to contain a splice acceptor sequence where the tenth and eleventh positions are, respectively, A and G, the ninth position is T, and four additional bases—those in the first, second, fourth and twelfth positions—are identical to a base in the corresponding position of CTYCYYYNTAGG, which is one of the sequences represented by SEQ ID NO:4 (yyyyyyyntagG).

The nucleic acid molecule of the present invention may be further engineered to reduce the number of CG (“CpG”) dinucleotides, in order to minimize the risk of inactivation of the C31-Int transgene through methylation by DNA-cytosine-5-methyltransferase at CpG dinucleotides (Pfeifer et al., EMBO J. 4:2879-2884, 1985). In general, the number of CpG dinucleotides is reduced as much as possible while still maintaining a preferred codon composition (e.g, at least 50% of C31-Int codons are optimal for the eukaryotic host). Reduction of CpG dinucleotides generally occurs through introduction of silent mutations, which most preferably replace the optimal codon with the second most prevalent codon, and may also replace the optimal codon with the third or fourth most prevalent codon. For an optimized C31-Int gene sequence, the number of CpG dinucleotides is preferably reduced by at least 40%, more preferably by at least 70% and most preferably by at least 90-100%. In preferred embodiments, the codon optimized C31-Int nucleic acid molecule comprises fewer than 200, 150, 100, or 50 CpG dinucleotides, or comprises no CpG dinucleotides. The codon optimized C31-Int may also be engineered to specifically eliminate “immuno-stimulatory” CpG motifs, which comprise the sequence RRCGYY. In one embodiment, the C31-Int nucleic acid molecule of the present invention does not comprise the sequence RRCGYY. In a further embodiment, a C31-Int gene that has been codon optimized, and has been engineered to reduce potential splice sites and CpG motifs, has the sequence presented in SEQ ID NO:5.

Other modifications may be made to further engineer the C31-Int gene for optimal expression of the C31-Int protein. In one embodiment, the C31-Int gene is engineered with a Kozak consensus sequence that spans the translational start codon. Kozak consensus sequences are generally represented by the sequence: GCCRCCATGG, in which the “ATG” represents the translational start codon, and may differ according to species (see, e.g., Kozak M, Cell 44:283-92, 1986; Kozak M, Nucleic Acids Res 15:8125-48, 1987; Kozak M, J Cell Biol 108:229-241, 1989; Jacobs G H et al., Nucleic Acids Res 30:310-1, 2002).

In another embodiment, the C31-Int gene also comprises sequence encoding a nuclear localization signal (NLS) to facilitate the import of cytoplasmic proteins into the nucleus (see, e.g., Gorlich et al., Science 271:1513-1518, 1996). Numerous NLS sequences, which share a high proportion of basic amino acids, have been characterized (see, e.g., Boulikas, Crit. Rev. Eucar. Gene Expression 3:193-227, 1993); a prototypical NLS of seven amino acids is derived from the T-antigen of the SV40 virus (Kalderon et. al, Cell, 39, 499-509 (1984)). Exemplary C31-Int genes comprising C-terminal NLS sequences are provided in SEQ ID NO:8 and SEQ ID NO:13, as further described in the Examples.

In yet another embodiment, the C31-Int gene is engineered with a second translational termination codon positioned 3′ to the first translational termination codon, preferably immediately 3′ thereto, and 5′ to the polyadenylation signal. This second stop codon is added to ensure proper translational termination.

A nucleic acid of the present invention encodes a C31-Int that is functionally active and is capable of catalyzing recombination at C31-Int recognition sequences in a eukaryotic host cell. As used herein, a C31-Int “that catalyzes recombination at phiC31 recognition sequences in the eukaryotic host cell” is one that is capable of catalyzing recombination at the recognition sequences. C31-Int recognition sequences, designated “attP” and “attB” are known in the art (Thorpe et al. Proc. Natl. Acad. Sci. USA, 95, 5505-5510 (1998)). Minimal recognition sequences have also been described (Groth et al., Proc. Natl. Acad. Science, 97, 5995-6000 (2000)). A functionally active C31-Int may catalyze recombination at any site known to be recognized by the native C31-Int.

A functionally active C31-Int generally has the protein sequence presented in SEQ ID NO:2. However, one or more changes in the amino acid sequence may be made without eliminating recombinase activity. Such changes are usually conservative. A conservative amino acid substitution is one in which an amino acid is substituted for another amino acid having similar properties such that the folding or activity of the protein is not significantly affected. Aromatic amino acids that can be substituted for each other are phenylalanine, tryptophan, and tyrosine; interchangeable hydrophobic amino acids are leucine, isoleucine, methionine, and valine; interchangeable polar amino acids are glutamine and asparagine; interchangeable basic amino acids are arginine, lysine and histidine; interchangeable acidic amino acids are aspartic acid and glutamic acid; and interchangeable small amino acids are alanine, serine, threonine, cysteine and glycine.

A variety of systems for determining whether a codon optimized C31-Int retains functional recombinase activity are known in the art and include systems for directly assessing the nucleic molecules that were recombined, as well as indirectly assessing recombinase activity through, for instance, a reporter gene which is activated, inactivated, or eliminated as a result of recombination. Such experiments may use cell-free systems comprising all the necessary components for recombination, or may use cultured cells or transgenic animals that have been engineered to express the C31-Int. Exemplary systems are further described in the Examples 2 and 3. A functionally active C31-Int preferably catalyzes recombination at least as efficiently as the wild type C31-Int, and more preferably catalyzes recombination at a higher level than the wild-type C31-Int.

Furthermore, a functionally active C31-Int encoded by an optimized C31-Int gene is preferably expressed at levels that are at least comparable to and more preferably higher than those encoded by a native C31-Int gene. The term “expression” refers to both transcription (mRNA levels) and translation (protein levels). Thus, in one embodiment it is preferred that in a given host, a codon-optimized C31-Int gene is transcribed at a comparable or higher level than a wild-type C31-Int gene, assuming that transcription of both is directed by essentially the same regulatory sequences. In another embodiment it is preferred that in a given host, protein expression from a codon optimized C31-Int gene is comparable to or higher than that from a wild-type C31-Int gene, assuming that the corresponding mRNAs are expressed at essentially comparable levels. Methods for analyzing mRNA and protein expression are well known in the art. For instance, Northern blotting, slot blotting, ribonuclease protection, or quantitative RT-PCR (e.g., using the TaqMan®, PE Applied Biosystems) may be used to assess mRNA expression (e.g., Current Protocols in Molecular Biology (1994) Ausubel F M et al., eds., John Wiley & Sons, Inc., chapter 4; Freeman W M et al., Biotechniques (1999) 26:112-125). Protein expression may be monitored with specific antibodies or antisera directed against either the C31-Int protein or specific peptides. A variety of means, including Western blotting, ELISA, or in situ detection, are available (Harlow E and Lane D, 1988, Antibodies: A Laboratory Manual, CSH Laboratory Press, New York).

An engineered C31-Int gene may differ in its methylation status (i.e., the proportion of methylated CpG dinucleotides) from a native C31-Int gene. Thus, further assessment of an engineered C31-Int gene may include detection of its methylation status, which is typically performed by Southern analysis and shows certain enzymes' inability to digest methylated DNA.

The present invention is directed to nucleic acid molecules comprising optimized C31-Int genes. Codon optimized C31-Int nucleic acid molecules may be generated by any available means. In one example, the generation of the synthetic gene involves annealing oligonucleotides to generate small subfragments, ligation of these subfragments to generate larger fragments, and eventual ligation of the full-length gene fragment (Scrable and Stambrook, Genetics 147, 297-304 (1997); EP1005574). As used herein, the term “genetic engineering” refers to any method of generating a nucleic acid molecule that differs from the corresponding native nucleic acid molecule. Accordingly, a genetically engineered nucleic acid molecule encoding a C31-Int is one that has been produced through human manipulation. A C31-Int gene may be inserted in a cloning vector, including bacteriophages such as lambda derivatives, or plasmids such as PBR322, pUC plasmid derivatives and the Bluescript vector (Stratagene, San Diego, Calif.). A C31-Int gene can be inserted into any appropriate expression vector for the transcription and translation of the inserted protein-coding sequence. Exemplary expression vectors are further described in the Examples. A variety of host-vector systems may be utilized to express the protein-coding sequence such as mammalian cell systems infected with virus (e.g. vaccinia virus, adenovirus, etc.); insect cell systems infected with virus (e.g. baculovirus); microorganisms such as yeast containing yeast vectors, or bacteria transformed with bacteriophage, plasmid or cosmid DNA. The present invention encompasses vectors comprising an optimized C31-Int gene, as well as microorganisms transformed with such vectors. A preferred microorganism is E. coli.

The present invention is further directed to cultured eukaryotic cells and non-human transgenic organisms that harbor a nucleic acid molecule comprising an optimized C31-Int gene in their genomes. Non-native nucleic acid is introduced into cultured cells or non-human laboratory animals by any expedient method. Preferred cultured cells are vertebrate cells, particularly those derived from mouse, rat, human, rabbit, or zebrafish. Methods for generating transformed cells are known in the art and include transfection, electroporation, particle bombardment, viral or retroviral infection, etc. Preferred transgenic animals are mammals, particularly mice or rats, and teleost, such as zebrafish. Methods of making transgenic non-human organisms are well-known in the art (see, e.g., for mice: Brinster et al., Proc. Nat. Acad. Sci. USA 1985, 82:4438-42; U.S. Pat. Nos. 4,736,866, 4,870,009, 4,873,191, 6,127,598; Hogan, B., Manipulating the Mouse Embryo, Cold Spring Harbor Laboratory Press, Cold Spring Harbor, N.Y., 1986; for rats: Murphy, D. and Carter, D. A. (1993) Transgenesis Techniques, Principles and Protocols, Humana, Totowa, N.J.; Mullins L J and Mullins J J, J Clin Invest 97:1557-60, 1996; for zebrafish: Lin S, Methods Mol Biol 136:375-383, 2000; Linney et al., Dev Biol 213:207-16, 1999; Ju et al., Dev Genet 25:158-67, 1999).

Cultured cells and transgenic animals of this invention, which comprise an optimized C31-Int gene in their genome, may further comprise C31-Int recognition sequences, also known as “att” sequences. Typically, for the production of doubly transgenic animals, two transgenic animal strains are generated, one comprising the recombinase gene and the other comprising recognition sequences, and the two components are brought into the same animal by crossing. Methods for using recombinase systems (i.e., a recombinase and associated recognition sites) for studying gene function are well known in the art (see, e.g., Rajewsky et al., J. Clin. Invest., 98,:600-603, 1996; Nagy, Genesis, 26:99-109,2000). The system comprising the C31-Int gene and its recognition sequences provides for the controlled activation or inactivation of genes of interest, and accordingly, methods for studying the function of such genes. When the recognition sites flank a particular gene of interest, expression of the recombinase can effect elimination (knock-out) of that gene in host cells. If the recognition sites are placed in inverted orientation, the flanked DNA sequence can be inverted. Alternatively, if the recognition sequences flank a sequence that interrupts a gene of interest, expression of the recombinase can effect activation of that gene by eliminating the disrupting sequence. Furthermore, the att-flanked DNA sequence can be exchanged for a different att-flanked sequence that is co-introduced with the C31-Int. In certain embodiments, the recombinase is expressed under the control of tissue- or temporal-specific promoters, such that the gene of interest is specifically activated or inactivated at particular developmental time points or only in particular tissues. The recombinase may also be expressed under the control of regulatory elements that are specifically activated in response to external agents, such as a hormone, an antibiotic (e.g., tetracycline), etc.

All references cited herein, including patents, patent applications, and publications, are herein incorporated by reference in their entireties.

### EXAMPLES

### Example 1

### Design of a C31-Int Gene for Expression in Mouse Cells

A version of a C31-Int gene that had been previously engineered to include sequence encoding a nuclear localization signal, designated C31-Int (CNLS), was further engineered for expression in mouse cells. The original nucleic acid sequence encoding C31-Int (CNLS) is presented in SEQ ID NO:6, and the corresponding protein sequence is presented in SEQ ID NO:7.

To design a C31-Int (CNLS) gene sequence with optimal properties for expression in the mouse, silent mutations were introduced into the wild type coding sequence (SEQ ID NO:6), and codons for particular amino acids were changed to the optimal codons for those amino acids in the mouse, as presented in Tables 1 and 4 (below).

In the next step, this codon-optimized sequence was analyzed for motifs matching either the splice donor or the splice acceptor consensus sequences. Sequences matching the 5′ splice donor consensus were found at four positions in the codon-optimized C31-Int gene. To eliminate these potential splice sites, these four sequences were changed to sequences less favorable for splicing through silent mutations in which the optimal codon was replaced with the second most prevalent codon (see Tables 1 and 4). Sequences matching the 3′ splice acceptor consensus were found at two positions in the gene and were changed to sequences less favorable for splicing through silent mutations replacing the optimal codon with the second or third most prevalent codon. Table 2 shows the silent mutations introduced to eliminate potential splice sites from the codon-optimized C31-Int(CNLS) gene. The “consensus sequences,” as shown for the 5′ splice donor and the 3′ splice acceptor sites, refer to motifs that were present in an “intermediate sequence” derived following codon-optimization of the C31-Int(CNLS) gene (and may therefore not be present in the C31-Int(CNLS) nucleotide sequence presented in SEQ ID NO:6). The “modified sequences” have incorporated the silent mutations and are present in the optimized C31-Int gene presented in SEQ ID NO:8. Numbering of nucleotides refers to the position within the C31-Int(CNLS) gene, the A of the ATG start codon being +1. Nucleotides that were altered are underlined. Capital letters refer to exon sequence, and lower case letters refer to intron sequence. Sequences are shown with nucleotides grouped according to codons.

TABLE 2
Silent mutations introduced to eliminate potential splice sites.

| Elimination of 5′ splice donor consensus | | | Elimination of 3′ splice acceptor consensus | | |
|---|---|---|---|---|---|
| Position of 5′ | Consensus | Modified | Position of 3′ | Consensus | Modified |
| consensus | sequence | sequence | consensus | sequence | sequence |
| 316-324 | AAG gtg atg | AAG gtc atg | 94-105 | gcc acc cag agG | c gcc acc cag agA |
| 337-345 | ATC gtg agc | ATC gtg tcc | 868-879 | agg gac ccc agG | g agg gac ccc cgG |
| 553-561 | CTG gtg agc | CTC gtg agc | | | |
| 1179-1187 | C AGg tgc ag | C AGa tgc ag | | | |

Through this codon-optimization process, the number of CpG methylation sites was simultaneously reduced. To further reduce the number of CpG dinucleotides and in parallel eliminate immuno-stimulatory CpG motifs from the gene sequence, the CpG dinucleotide motif was altered at 20 positions matching the consensus for immuno-stimulatory CpGs (RRCGYY), as shown in Table 3. To eliminate the CpG dinucleotide, individual codons were replaced by the second most prevalent codon for the particular amino acid. Table 3 shows the silent mutations introduced to eliminate potential immuno-stimulatory CpG motifs. As in Table 2, the “consensus sequences,” refer to motifs that were present in an “intermediate sequence” derived following codon-optimization of the C31-Int(CNLS) gene, and the “modified sequences” are present in the optimized C31-Int gene presented in SEQ ID NO:8. Numbering of nucleotides refers to the position within the C31-Int(CNLS) gene, the A of the ATG start codon being +1. Nucleotides that were altered are underlined. Sequences are shown with nucleotides grouped according to codons.

TABLE 3
Silent mutations introduced to eliminate CpG motifs.

| Position of RRCGYY | Consensus | Modified |
|---|---|---|
| consensus | sequence | sequence |
| 40-45 | ggc gcc | gga gcc |
| 79-84 | agc gcc | tcc gcc |
| 106-111 | agc gcc | tcc gcc |
| 205-210 | agc gcc | tcc gcc |
| 235-330 | gac gcc | gat gcc |
| 442-447 | gac gcc | gat gcc |
| 472-477 | agc gcc | tcc gcc |
| 778-783 | gac gcc | gat gcc |
| 784-789 | gac gcc | gat gcc |
| 832-837 | agc gcc | tcc gcc |
| 1099-1104 | agc gcc | tcc gcc |
| 1129-1134 | ggc gcc | gga gcc |
| 1210-1215 | agc gcc | tcc gcc |
| 1420-1425 | gac gcc | gat gcc |
| 1429-1434 | aac gcc | aat gcc |
| 1465-1470 | ggc gcc | gga gcc |
| 1537-1542 | ggc gcc | gga gcc |
| 1612-1617 | gac gcc | gat gcc |
| 1618-1623 | gac gcc | gat gcc |
| 1807-1812 | gac gcc | gat gcc |

Overall, the number of CpG dinucleotides was reduced from 245 in the wild type C31-Int(CNLS) gene (SEQ ID NO:6) to 132 in the codon-optimized gene (SEQ ID NO:8).

To further improve expression, the sequence GCCACC was attached 5′ to the ATG start codon in order to generate a close match to the Kozak consensus sequence GCCRCCATGG. A second stop codon (TGA) was added at the 3′ terminus of the coding sequence to ensure proper translational termination.

Finally, the gene was flanked by restriction enzyme (EcoRV) sites for cloning purposes. The sequence nucleotide sequence of the optimized C31-Int gene, designated C31-Int(CNLS)-CO, is provided in SEQ ID NO:8.

The engineered gene was synthesized by GeneArt (Regensburg, Germany).

Table 4 shows the codon usage for each amino acid in the wild type version of C31-Int(CNLS) and C31-Int(CNLS)-CO, as well as codon usage in phiC31 and in mouse. Number of occurrences (#) and frequencies per thousand(/1000) are displayed.

TABLE 4
Codon frequencies in C31-Int(CNLS) and C31-Int(CNLS)-CO

| | | phiC31 | mus | | | C31-Int(CNLS)- | |
|---|---|---|---|---|---|---|---|
| Amino | | phage/ | musculus/ | C31-Int(CNLS) | | CO | |
| acid | triplet | 1000 | 1000 | # | /1000 | # | /1000 |
| ARG | CGA | 5 | 6.7 | 3 | 4.8 | 0 | 0.0 |
| | CGC | 29.2 | 10.1 | 25 | 40.3 | 0 | 0.0 |
| | CGG | 21.3 | 10.5 | 14 | 22.5 | 1 | 1.6 |
| | CGU | 12.8 | 4.7 | 5 | 8.1 | 0 | 0.0 |
| | AGA | 0.7 | 11.4 | 0 | 0.0 | 3 | 4.8 |
| | AGG | 1.3 | 11.6 | 6 | 9.7 | 49 | 78.9 |
| Leu | CUA | 2.2 | 7.5 | 0 | 0.0 | 0 | 0.0 |
| | CUC | 14.9 | 19.6 | 9 | 14.5 | 1 | 1.6 |
| | CUG | 27.2 | 39.3 | 12 | 19.3 | 42 | 67.6 |
| | CUU | 24.3 | 12.3 | 18 | 29.0 | 0 | 0.0 |
| | UUA | 0 | 6 | 0 | 0.0 | 0 | 0.0 |
| | UUG | 6.1 | 12.5 | 4 | 6.4 | 0 | 0.0 |
| SER | UCA | 2.6 | 11.2 | 2 | 3.2 | 0 | 0.0 |
| | UCC | 12.5 | 18.1 | 4 | 6.4 | 8 | 12.9 |
| | UCG | 19.4 | 4.5 | 16 | 25.8 | 0 | 0.0 |
| | UGU | 3.7 | 15.4 | 2 | 3.2 | 0 | 0.0 |
| | AGC | 11.6 | 20 | 8 | 12.9 | 25 | 40.3 |
| | AGU | 3.5 | 12.2 | 1 | 1.6 | 0 | 0.0 |
| THR | ACA | 2 | 15.6 | 2 | 3.2 | 0 | 0.0 |
| | ACC | 18.5 | 19.6 | 9 | 14.5 | 37 | 59.6 |
| | ACG | 39.5 | 6.1 | 21 | 33.8 | 0 | 0.0 |
| | ACU | 8 | 13.2 | 5 | 8.1 | 0 | 0.0 |
| PRO | CCA | 1 | 17.3 | 1 | 1.6 | 0 | 0.0 |
| | CCC | 1.2 | 19.1 | 9 | 14.5 | 33 | 53.1 |
| | CCG | 32.4 | 6.8 | 18 | 29.0 | 0 | 0.0 |
| | CCU | 5.2 | 18.7 | 5 | 8.1 | 0 | 0.0 |
| ALA | GCA | 9.5 | 15.5 | 7 | 11.3 | 0 | 0.0 |
| | GCC | 49.2 | 26.7 | 24 | 38.6 | 65 | 104.7 |
| | GCG | 45.6 | 7.1 | 27 | 43.5 | 0 | 0.0 |
| | GCU | 18.7 | 19.7 | 7 | 11.3 | 0 | 0.0 |
| GLY | GGA | 7 | 17.3 | 5 | 8.1 | 4 | 6.4 |
| | GGC | 44.5 | 22.9 | 25 | 40.3 | 47 | 75.7 |
| | GGG | 17.5 | 15.9 | 19 | 30.6 | 0 | 0.0 |
| | GGU | 17.1 | 11.7 | 2 | 3.2 | 0 | 0.0 |
| VAL | GUA | 3.8 | 6.9 | 4 | 6.4 | 0 | 0.0 |
| | GUC | 29.9 | 15.6 | 17 | 27.4 | 1 | 1.6 |
| | GUG | 29.4 | 29 | 8 | 12.9 | 37 | 59.6 |
| | GUU | 17.6 | 10.1 | 9 | 14.5 | 0 | 0.0 |
| LYS | AAA | 1.3 | 21.3 | 2 | 3.2 | 1 | 1.6 |
| | AAG | 45.5 | 34.6 | 39 | 62.8 | 40 | 64.4 |
| ASN | AAC | 24.7 | 21.5 | 11 | 17.7 | 12 | 19.3 |
| | AAU | 3.5 | 15.5 | 2 | 3.2 | 1 | 1.6 |
| GLN | CAA | 3.1 | 11.6 | 6 | 9.7 | 0 | 0.0 |
| | CAG | 20.4 | 34.5 | 13 | 20.9 | 19 | 30.6 |
| HIS | CAC | 15 | 15.3 | 9 | 14.5 | 10 | 16.1 |
| | CAU | 2.8 | 9.9 | 1 | 1.6 | 0 | 0.0 |
| GLU | GAA | 26.7 | 26.9 | 26 | 41.9 | 1 | 1.6 |
| | GAG | 34.9 | 40.3 | 26 | 41.9 | 51 | 82.1 |
| ASP | GAC | 63.1 | 27.5 | 40 | 64.4 | 32 | 51.5 |
| | GAU | 6.6 | 21.4 | 1 | 1.6 | 9 | 14.5 |
| TYR | UAC | 21.5 | 16.8 | 10 | 16.1 | 12 | 19.3 |
| | UAU | 5.7 | 11.8 | 2 | 3.2 | 0 | 0.0 |
| CYS | UGC | 8.2 | 12.6 | 5 | 8.1 | 7 | 11.3 |
| | UGU | 2.4 | 10.9 | 2 | 3.2 | 0 | 0.0 |
| PHE | UUC | 27.4 | 21.6 | 19 | 30.6 | 19 | 30.6 |
| | UUU | 2.7 | 16 | 0 | 0.0 | 0 | 0.0 |
| ILE | AUA | 0.9 | 6.6 | 0 | 0.0 | 0 | 0.0 |
| | AUC | 19.5 | 22.9 | 18 | 29.0 | 31 | 49.9 |
| | AUU | 14.1 | 14.6 | 13 | 20.9 | 0 | 0.0 |
| MET | AUG | 18.6 | 22.2 | 11 | 17.7 | 11 | 17.7 |
| TRP | UGG | 19.3 | 12.9 | 11 | 17.7 | 11 | 17.7 |
| TER | UAA | 0.7 | 0.6 | 0 | 0.0 | 0 | 0.0 |
| | UAG | 0.8 | 0.5 | 1 | 1.6 | 0 | 0.0 |
| | UGA | 2.7 | 1.1 | 0 | 0.0 | 1 | 1.6 |

A second codon optimized C31-Int gene, designated “C31-Int(CNLS)-CO-delCG,” was designed and generated (synthesized by GeneArt [Regensburg, Germany]); its sequence is presented in SEQ ID NO:13. Like the C31-Int(CNLS)-CO (SEQ ID NO:8), it comprises a Kozak consensus sequence, a second stop codon, and sequence encoding a carboxy-terminal NLS. However, C31-Int(CNLS)-CO-delCG was specifically engineered to eliminate all CG dinucleotides from its coding sequence.

### Example 2

### Functional Analysis of C31-Int(CNLS)-CO

In order to test the activity of the codon-optimized form of C31-Int(CNLS) (“C31-Int(CNLS)-CO”) in mouse cells, the activities of expression vectors comprising C31-Int(CNLS) and C31-Int(CNLS)-CO genes were compared.

A. Description of Plasmids

The C31-Int(CNLS) expression vector, whose sequence is presented in SEQ ID NO:9, was designated pCMV-C31-Int(CNLS) and was generated using the C31-Int gene sequence amplified from phage DNA (DSM-49156, DSMZ-Deutsche Sammlung von Mikroorganismen und Zellkulturen GmbH, Mascheroder Weg 1b, D-38124 Braunschweig, Germany). The stop codon of the native C31-Int sequence was replaced by a 21 bp sequence encoding the 7 amino acid SV40 T-antigen NLS (PKKKRKV; Kalderon et al., 1984), followed by a new stop codon. pCMV-C31-Int(CNLS) comprises the following sequence: a 700 bp cytomegalovirus immediate early gene promoter (position 12-711), a 270 bp hybrid intron (position 712-981), the NLS-modified C31-Int gene (position 989-2851), and a 189 bp synthetic polyadenylation sequence (position 2854-3043).

The C31-Int(CNLS)-CO expression vector, whose sequence is presented in SEQ ID NO: 10, was designated pCMV-C31-Int(CNLS)-CO and is similar to pCMV-C31-Int(CNLS), except that it comprises C31-Int(CNLS)-CO instead of C31-Int(CNLS).

Activities of the expression vectors pCMV-C31-Int(CNLS) and pCMV-C31-Int(CNLS)-CO were tested in reporter cells that contained a stably integrated “substrate vector,” comprising a beta-galactosidase coding sequence under control of an upstream SV40 promoter. The coding sequence was separated from the promoter and functionally disrupted by insertion of a 1.1 kb puromycin gene cassette (containing a stop codon and a polyadenylation sequence), and the cassette was flanked by C31-Int recognition sequences (5′, the 84 bp attB, and 3′, the 84 bp attP) adjacent to direct repeat loxP sites. Upon either C31-Int- or Cre-mediated recombination, the termination cassette would be deleted, allowing expression of beta-galactosidase.

The substrate vector, whose sequence is presented in SEQ ID NO: 11, was designated “pRK64” and was generated using the PSV-Pax1 vector backbone (Buchholz et al., Nucleic Acids Res. 24:4256-4262,1996).

All vectors were generated using standard molecular biology and recombinant DNA cloning techniques and their nucleotide sequences confirmed by DNA sequence analysis.

B. Cell Culture and Transfections:

To generate a stably transfected reporter cell line, 2.5×10⁶ NIH-3T3 cells (Andersson et al., Cell, 16, 63-75 (1979); DSMZ#ACC59; DSMZ-Deutsche Sammlung von Mikroorganismen und Zellkulturen GmbH, Mascheroder Weg 1b, D-38124 Braunschweig, Germany) were electroporated with 5 μg pRK64 plasmid DNA linearized with ScaI and plated into 10 cm petri dishes. The cells were grown in DMEM/Glutamax medium (Life Technologies) supplemented with 10% fetal calf serum at 37° C., 10% CO₂ in humid atmosphere, and passaged upon trypsinization. Two days after transfection the medium was supplemented with 1 mg/ml of puromycin (Calbiochem) for the selection of stable integrants. Resistant colonies were isolated and individually expanded in the absence of puromycin.

Standard Southern blotting methods were used to demonstrate stable integration of the transfected vector in puromycin-resistant clones. Briefly, genomic DNA from individual clones was prepared according to standard methods and 5-10 μg was digested with EcoRV. Digested DNA was separated in a 0.8% agarose gel and transferred to nylon membranes (GeneScreen Plus, NEN DuPont) under alkaline conditions for 16 hours. The filter was dried and hybridized for 16 hours at 65° C. with a P32-labeled probe representing the 5′ part of the E. coli beta-galactosidase gene. Hybridization was performed in a buffer containing 10% dextranesulfate, 1% SDS, 50 mM Tris and 100 mM NaCl, pH 7.5). After hybridization, the filter was washed with 2×SSC/1%SDS and exposed to BioMax MS1 X-ray films (Kodak) at −80° C.

A clone designated 3T3(pRK64)-3, which showed stable integration of pRK64, was selected for further analysis. To allow direct comparison of pCMV-C31-Int(CNLS) and pCMV-C31-Int(CNLS)-CO, the same amounts of these plasmids were introduced into 3T3(pRK64)-3 by transient transfection. Transfections were performed using the FuGene6 transfection reagent (Roche Diagnostics GmbH, Mannheim, Germany), essentially according to the manufacturers protocol. One day prior to transfections, approximately 10⁶ cells were plated into a 48-well plate. The next day, each well received 250 μl medium containing 200 ng plasmid DNA complexed to the FuGene6 transfection reagent. The 200 ng DNA preparations contained 50 ng of the luciferase expression vector pUHC13-1 (Gossen et al., Proc Natl Acad Sci USA., 89 5547-5551 (1992)), 4-32 ng of either pCMV-C31-Int(CNLS) or pCMV-C31-Int(CNLS)-CO, and pUC19 plasmid (GenBank#X02514; New England Biolabs Inc, Beverly, Mass.) to bring the total amount of DNA to 200 ng. The control sample contained 50 ng of pUHC13-1 and 150 ng pUC19. All samples contained a fixed amount of pUHC13-1 so that luciferase activity could be used to control for experimental variation of transfection and lysis. Individual preparations were tested in four replicate wells. One day after the addition of the DNA preparations, each well received additional 250 μl of growth medium. The cells of each well were lysed 48 hours after transfection with 100 μl lysis reagent supplemented with protease inhibitors (Roche Diagnostics) and centrifuged.

C. Enzyme Assays and Results

Enzyme (beta-galactosidase and luciferase) assays were performed using 20 μl lysate.

Recombination activity was measured as the level of beta-galactosidase activity (“Gal”). The beta-galactosidase chemiluminescence assay (Roche Diagnostics) was performed essentially according to the manufacturers' protocol in a Lumat LB 9507 luminometer (Berthold).

Values from the beta-galactosidase activity were normalized by luciferase activity (“Luc”). To measure luciferase activity, 20 μl lysate was diluted into 250 μl assay buffer (50 mM glycylglycin, 5 mM MgCl₂, 5 mM ATP), and the “Relative Light Units” (RLU) were counted in a Lumat LB 9507 luminometer after addition of 100 μl of a 1 mM luciferin solution (Roche Diagnostics). The mean and standard deviation for beta-galactosidase and luciferase RLU values corresponding to each DNA preparation were calculated from individual values for each of the four replicate wells. For each DNA sample, the RLU value of beta-galactosidase activity was divided by the RLU value for luciferase activity and multiplied by a factor of 10⁵. Values for enzyme activity are provided +/− standard deviations. Results of the recombination assay are shown in Table 5.

TABLE 5
Results of the recombination assay.

| | | Enzyme Activity |
|---|---|---|
| Plasmid transfected (ng) | | (RLU × 10⁵ |
| pCMV-C31-Int(CNLS) | pCMV-C31-Int(CNLS)-CO | [Gal/Luc]) |
| 0 | 0 | 328 +/− 281 |
| 4 | 0 | 1504 +/− 487 |
| 8 | 0 | 3085 +/− 596 |
| 16 | 0 | 3787 +/− 719 |
| 32 | 0 | 5157 +/− 798 |
| 0 | 4 | 1347 +/− 228 |
| 0 | 8 | 2453 +/− 558 |
| 0 | 16 | 3897 +/− 714 |
| 0 | 32 | 4201 +/− 715 |

As shown in Table 5, the transfections with C31-Int(CNLS)-CO and C31-Int(CNLS) expression vectors resulted in comparable levels of beta-galactosidase activity. The values for 16 ng and 32 ng DNA amounts were close to saturation of the test system, as the doubling of the DNA amounts resulted only in a minor increase of recombinase activity. It was concluded that the codon-optimized C31-Int(CNLS) gene was fully functional in mammalian cells.

### Example 3

### Generation of Transgenic Mice Comprising the C31-Int-CO Gene

To test whether the C31-Int(CNLS)-CO gene confers enhanced C31 activity in transgenic mice, either the C31-Int(CNLS) gene or the C31-Int(CNLS)-CO gene was expressed from the identical locus in the mouse genome. The genes were inserted downstream of the ROSA26 promoter (Genbank entry gi:1778857) through homologous recombination in ES cells and chimaeric mice were generated from the recombined ES cells. These mice were mated to reporter mice carrying a C31 substrate reporter vector. Different tissues of offspring carrying the substrate vector plus one of the recombinase genes were then analysed for substrate recombination as indicated by LacZ expression.

A Vector Construction

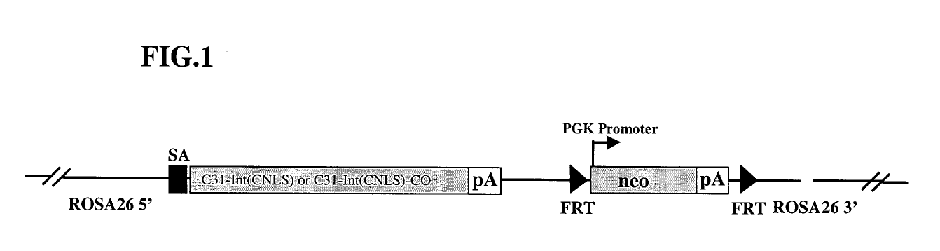

FIG. 1 shows the ROSA26 targeting vectors for C31-Int(CNLS) (Seq ID NO: 12) and C31-Int(CNLS)-CO (Seq ID NO: 13).

The C31-Int(CNLS) and C31-Int(CNLS)-CO coding sequences were inserted downstream of a splice acceptor site (SA) such that they were expressed from the endogenous ROSA26 promoter after homologous recombination in ES cells. The coding regions were followed by a polyadenylation signal (pA) for proper transcriptional termination. An FRT-flanked selection marker conferring resistance to G418 (PGK-neo-pA) was inserted downstream. The constructs were flanked by 5′ and 3′ ROSA26 homology arms for homologous recombination in ES cells.

To generate a targeting vector for the ROSA26 locus (Friedrich, G A. and Soriano, P. (1991) Genes Dev. 5, 1513-1523), a 129 Sv/Ev-BAC library (Incyte Genomics) was screened with a probe against exon2 of the Rosa26 locus (amplified from mouse genomic DNA by PCR using Rscreen1s (SEQ ID NO:14) and Rscreen1as (SEQ ID NO:15) as primers). A BAC clone was identified, and an 11 kb EcoRV subfragment, containing the exons of the ROSA26 gene was subcloned. Of this subclone, two fragments, a 1 kb SacII/XbaI fragment (SEQ ID NO:16) and a 4 kb XbaI-fragment (SEQ ID NO:17) were used as upstream and downstream homology arms, respectively, and inserted into a vector comprising a FRT-flanked neomycin resistance gene. A splice acceptor from adenovirus site was inserted between the two homology arms and the resulting vector was designated pROSA12 (SEQ ID NO:18).

To generate the ROSA26 targeting vector for C31-Int(CNLS), the C31-Int(CNLS) coding region was inserted into pROSA12 (SEQ ID NO:18). The resulting vector was designated pROSA-SA-C31-Int(CNLS) (SEQ ID NO:12), which contained the following features as depicted in FIG. 1: an upstream homology arm for homologous recombination with the ROSA26 locus, a splice acceptor from adenovirus, the C31-Int(CNLS) coding sequence, a polyadenylation site, an FRT-flanked G418 selection cassette and the downststream ROSA26 homology arm.

The targeting vector for the C31-Int(CNLS)-CO gene was designated pROSA-SA-C31-Int(CNLS)-CO (SEQ ID NO:13) and carries the same features as the C31-Int(CNLS) targeting vector with the exception that the coding region for C31-Int(CNLS) was replaced by the C31-Int(CNLS)-CO coding region.

B. Homologous Recombination in ES Cells

The ES cell line C57B16 (Eurogentec, Belgium) was grown on mitotically inactivated feeder layer (Mitomycin C (Sigma M-0503)) comprised of mouse embryonic fibroblasts in the medium of 1×DMEM high Glucose (Invitrogen 41965-062), 2 mM Glutamin (Invitrogen 25030-024), 1× Non Essential Amino Acids (Invitrogen 11140-035) 1 mM Sodium Pyruvate (Invitrogen 11360-039), 0.1 mM β-Mercaptoethanol (Invitrogen 31350-010), 2×10⁶ u/L Leukemia Inhibitory Factor (Chemicon ESG 1107) and 20% fetal bovine serum (pre-tested for ES cell culture).

Vectors pROSA-SA-C31-Int(CNLS) or pROSA-SA-C31-Int(CNLS)-CO linearized with the restriction enzymes I-SceI or SacII, respectively, were introduced into the ES cells by electroporation. Rapidly growing cells were used one day after passaging. Upon trypsinization with 0.25% Trypsin-EDTA (Invitrogen 25200-056) cells were resuspended in PBS (Invitrogen 20012-019) and preplated for 25 min on gelatinized 10 cm plates to remove undesired feeder cells. The supernatant was harvested, ES cells were washed once in PBS and counted (Neubauer hemocytometer). 10⁷ cells were mixed with 30 μg of linearized vector in 800 μL of transfection buffer (20 mM Hepes, 137 mM NaCl, 15 mM KCl, 0.7 mM Na₂HPO₄, 6 mM Glucose 0.1 mM β-Mercaptoethanol in H₂O) and electroporated using a Biorad Gene Pulser with Capacitance Extender set on 240 V and 500 μF. Electroporated cells were seeded at a density of 2.5×10⁶ cells per 10 cm tissue culture dish onto a previously prepared layer of neomycin-resistant inactivated mouse embryonic fibroblasts.

48 h after electroporation, the medium was replaced on all dishes by medium containing 250 μg/ml Geneticin (Invitrogen 10131-019) for positive selection of G418 resistant ES clones.

On day 8 after electroporation, ES colonies were isolated as follows:

Medium was replaced by PBS and the culture dishes were placed on the stage of a binocular (Nikon SMZ-2B). Using low magnification (25×) individual ES clones of undifferentiated appearance were removed from the surface of the culture dish by suction into the tip of a 20 μl pipette (Eppendorf). The harvested clones were placed in individual wells of 96 well plates containing 30 μL of Trypsin. After disruption of the colonies by pipetting with a multichannel pipette (Eppendorf), cells were seeded onto feeder containing 96 well plates with pre-equilibrated complete ES-medium.

Cells were grown for 3 days with daily medium changes and then split 1:2 on gelatinized (Sigma G-1890) 96 well plates. Cells were lysed 3 days after splitting, genomic DNA was prepared and analysed by Southern blot for homologous recombination. For Southern blots, genomic DNA was digested with EcoRV and separated on a 0.8% agarose gel. After transfer to a positively charged nylon membrane the samples were hybridised to probe ROSA5.1 (SEQ ID NO:19) representing sequence of the ROSA26 locus upstream of the targeting vector. The homologous recombination event was indicated by the presence of a 3.8 kb band.

C. Generation of Mice

ES cell clones carrying a single copy of the targeting vector homologously recombined in their ROSA26 locus were injected into mouse blastocysts and subsequently transferred to pseudopregnant foster mothers in order to generate chimaeras.

Balb/C male and female mice (Janvier, France) were mated to obtain blastocysts from fertilized females. Plug positive females were set aside, and 3 days later blastocysts were isolated by flushing their uteri.

For microinjection, 5-6 blastocysts were placed in a drop of DMEM with 15% FCS under mineral oil. A flat tip, piezo actuated microinjection-pipette with an internal diameter of 12-15 μm was used to inject 15 ES cells into each blastocyst. After recovery, ten injected blastocysts were transferred to each uterine horn of 2.5 days post coitum, pseudopregnant NMRI females that had been mated with vasectomized males.

After birth of the mice, high percentage chimaeras were identified by coat color chimerism.

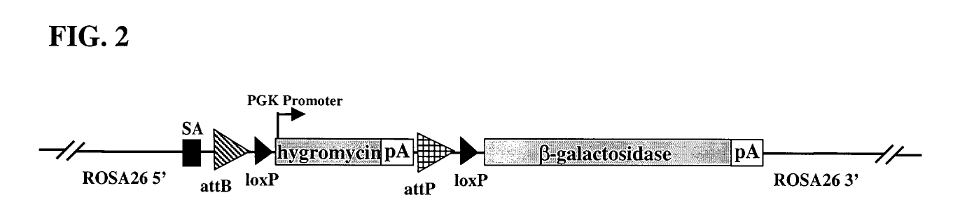

These chimaeras were mated to heterozygous C31 reporter mice carrying the C31 substrate reporter in the ROSA26 locus (SEQ ID NO:20). FIG. 2 shows the modified ROSA26 locus of C31 reporter mice. A recombination substrate has been inserted in the ROSA26 locus. The substate has a splice acceptor (SA) followed by a cassette containing the hygromycin resistance gene driven by a PGK promoter and flanked by the recombination sites attB and attP. In addition, the reporter contains two Cre recognition sites (loxP) in direct orientation next to the att sites. This cassette is followed by the coding region for β-galactosidase, which is only expressed when the hygromycin resistance gene has been deleted by recombination.

Offspring of the crosses were genotyped for the presence of the transgenes by the following PCR assays:

PCR for C31-Int(CNLS): primers C31-1 (SEQ ID NO: 21) and C31-2 (SEQ ID NO:22) amplifying a diagnostic fragment of 500 bp. The PCR reaction contained 5 μl 10×PCR buffer (Invitrogen), 2 μl 50 mM MgCl2, 1.5 μl 10 mM dNTP-mix, 2 μl (10 pmol) of each primer, 0.5 μl Taq-polymerase (5 U/μl) and water to a volume of 50 μl. The program used for the PCR reactions was: 94° C. for 30 s, 55° C. for 30 s and 72° C. for 1 min in 30 cycles.

PCR for C31-Int(CNLS)-CO: primers were C31-h-5′ (SEQ ID NO: 23) and C31-h-3′ (SeQ ID NO:24) amplifying a 500 bp diagnostic fragment of the C31-Int(CNLS)-CO gene. The PCR reaction contained 5 μl 10×PCR buffer (Invitrogen), 2 μl 50 mM MgCl₂, 1.5 μl 10 mM dNTP-mix, 10 pmol of each primer, 2.5 U Taq polymerase in a total volume of 50 μl. PCR was performed for 30 cycles of 94° C. for 30 s, 55° C. for 1 min and 72° C. for 1 min.

PCR for ROSA26-C31 reporter allele (LacZ gene): The PCR was performed using tail DNA and the primers β-Gal 3 (SEQ ID NO:25) and β-Gal 4 (SEQ ID NO:26) amplifying a diagnostic fragment of 315 bp. The PCR reaction contained 5 μl 10×PCR buffer (Invitrogen), 2.5 μl 50 mM MgCl₂, 2 μl 10 mM dNTP-mix, 1 μl (10 pmol) of each primer, 0.4 μl Taq-polymerase (5 U/μl) and water to a volume of 50 μl. The program used for the PCR reactions was: 94° C. for 1 min, 60° C. for 1 min and 72° C. for 1 min in 30 cycles.

Tissues from mice carrying either the ROSA-C31-Int(CNLS) or the ROSA-C31-Int(CNLS)-CO targeted to the ROSA26 locus as well as the C31 substrate reporter targeted to the second ROSA26 allele and from a control mouse carrying the reporter allele only were dissected. For wholemount staining, the tissues were rinsed in 0.1 M PB (0.1 M K₂HPO₄, pH 7.3) and then fixed in fixative (0.2% glutaraldehyde, 5 mM EGTA, 2 mM MgCl₂ in 0.1 M PB) for 45 min at room temperature. They were then washed three times for 15 min at room temperature in LacZ wash buffer (2 mM MgCl₂, 0.02% Nonidet-40 in 0.1 M PB). Subsequently, the tissues were stained for beta-galactosidase activity overnight at room temperature in X-Gal solution (1 mg/ml X-Gal (predissolved in DMSO), 4 mM potassium hexacyanoferrat III, 4 mM potassium hexacyanoferrat II, in LacZ wash buffer). Tissues were washed twice for 10 min at room temperature in LacZ wash buffer and pictures were taken.

D. Results

In double transgenic mice recombination activity of the C31-Int(CNLS) recombinase was measured through activity of the beta-galactosidase produced by the recombined but not the unrecombined C31 substrate reporter. Wholemounts of the corresponding tissues from a mouse carrying only the reporter substrate were used as control. In mice carrying the ROSA-C31-Int(CNLS) knock-in plus the reporter substrate, beta-gal activity indicating recombination could not be detected in any somatic tissues, but was detected in the gonads. In contrast, mice carrying the ROSA-C31-Int(CNLS)-CO knock-in plus the reporter showed high level recombination in all tissues analysed, visible by the dark blue color of the tissues, indicating recombination of the substrate, which led to the expression of β-galactosidase.

Since both C31 genes were expressed by the same promoter from the same chromosomal location, this difference provided evidence for the superior performance of the C31-Int(CNLS)-CO sequence when integrated into a eukaryotic genome in vivo.

What is claimed:

1. A genetically engineered nucleic acid molecule encoding a phiC31 integrase,
wherein the nucleic acid molecule comprises a nucleotide sequence optimized for expression in a eukaryotic host cell,
wherein the nucleotide sequence comprises at least 306 codons that are optimal for expression in the eukaryotic host cell, and
wherein the phiC31 integrase catalyzes recombination at phiC31 recognition sequences in the eukaryotic host cell.

2. The nucleic acid molecule of claim 1, wherein the nucleotide sequence comprises at least 430 codons that are optimal for expression in the eukaryotic host cell.

3. The nucleic acid molecule of claim 1, wherein the nucleotide sequence comprises at least 550 codons that are optimal for expression in the eukaryotic host cell.

4. The nucleic acid molecule of claim 1 wherein the nucleic acid encodes a protein comprising the amino acid sequence presented in SEQ ID NO:2.

5. The nucleic acid molecule of claim 1, wherein the eukaryotic host cell is from a species selected from the group consisting of mouse, rat, human, rabbit, and teleost.

6. The nucleic acid molecule of claim 1, wherein the nucleotide sequence does not contain a splice donor sequence selected from the group consisting of AAGgtaagt, AAGgtgagt, CAGgtaagt, and CAGgtgagt.

7. The nucleic acid molecule of claim 1, wherein the nucleotide sequence does not contain a splice donor sequence,
wherein the splice donor sequence consists of nine contiguous nucleotides,
wherein the fourth and fifth positions of the splice donor sequence consist of a G and a T, respectively, and
wherein at least five of the nucleotides in the first, second, third, six, seventh, eighth, and ninth positions are identical to the nucleotide in the corresponding position in any one of the sequences selected from the group consisting of AAGgtaagt, AAGgtgagt, CAGgtaagt, CAGgtgagt.

8. The nucleic acid molecule of claim 1, wherein the nucleotide sequence does not contain a splice donor sequence,
wherein the splice donor sequence consists of nine contiguous nucleotides,
wherein the fourth and fifth positions of the splice donor sequence consist of a G and a T, respectively, and
wherein at least four of the nucleotides in the first, second, third, six, seventh, eighth, and ninth positions are identical to the nucleotide in the corresponding position in any one of the sequences selected from the group consisting of AAGgtaagt, AAGgtgagt, CAGgtaagt, CAGgtgagt.

9. The nucleic acid molecule of claim 1, wherein the nucleotide sequence does not contain a splice donor sequence,
wherein the splice donor sequence consists of nine contiguous nucleotides,
wherein the fourth and fifth positions of the splice donor sequence consist of a G and a T, respectively, and
wherein at least three of the nucleotides in the first, second, third, six, seventh, eighth, and ninth positions are identical to the nucleotide in the corresponding position in any one of the sequences selected from the group consisting of AAGgtaagt, AAGgtgagt, CAGgtaagt, CAGgtgagt.

10. The nucleic acid molecule of claim 1, wherein the nucleotide sequence does not contain a splice acceptor sequence, wherein the splice acceptor sequence is selected from the group consisting of SEQ ID NO:3 and SEQ ID NO:4.

11. The nucleic acid molecule of claim 1, wherein the nucleotide sequence does not contain a splice acceptor sequence, wherein the splice acceptor sequence consists of 12 contiguous nucleotides,
wherein the ninth position of the nucleotide sequence of the splice acceptor consists of a C or a T,
wherein the tenth and eleventh positions of the nucleotide sequence of the splice acceptor consist an A and a G respectively,
and wherein at least five of the nucleotides in the first, second, third, fourth, fifth sixth, seventh, and twelfth positions are identical to the nucleotide in the corresponding position in any one of the sequences presented as SEQ ID NO:3 and SEQ ID NO:4.

12. The nucleic acid molecule of claim 1, wherein the nucleotide sequence does not contain a splice acceptor sequence, wherein the splice acceptor sequence consists of 12 contiguous nucleotides,
wherein the ninth position of the nucleotide sequence of the splice acceptor consists of a C or a T,
wherein the tenth and eleventh positions of the nucleotide sequence of the splice acceptor consist an A and a G, respectively, and
wherein at least four of the nucleotides in the first, second, third, fourth, fifth sixth, seventh, and twelfth positions are identical to the nucleotide in the corresponding position in any one of the sequences presented as SEQ ID NO:3 and SEQ ID NO:4.

13. The nucleic acid molecule of claim 1, wherein the nucleotide sequence comprises fewer than 200 CG dinucleotides.

14. The nucleic acid molecule of claim 13, wherein the nucleotide sequence comprises fewer than 150 CG dinucleotides.

15. The nucleic acid molecule of claim 13, wherein the nucleotide sequence comprises fewer than 100 CG dinucleotides.

16. The nucleic acid molecule of claim 13, wherein the nucleotide sequence comprises fewer than 50 CG dinucleotides.

17. The nucleic acid molecule of claim 13 wherein nucleotide sequence does not comprise the sequence RRCGYY.

18. The nucleic acid molecule of claim 13 having a coding sequence, wherein the nucleotide sequence does not comprise any CG dinucleotides in its coding sequence.

19. The nucleic acid molecule of claim 1 that has the sequence presented as SEQ ID NO:5.

20. The nucleic acid molecule of claim 1 having a translational start codon and a first translational termination codon, which further comprises at least one nucleotide sequence selected from the group consisting of:
a) a Kozak consensus sequence that spans the translational start codon,
b) a nucleotide sequence 3′ of the phiC31 integrase encoding region, which encodes a nuclear localization signal, and
c) a second translational termination codon positioned immediately 3′ to the first translational termination codon.

21. The nucleic acid molecule of claim 20 that has the sequence presented as SEQ ID NO:8 or SEQ ID NO:13.

22. The phiC31 integrase encoded by the nucleic acid molecule or any of claims 1, 6, 10, 13, 17, or 20.

23. A DNA vector comprising the nucleic acid presented in any of claims 1, 6, 10, 13, 17, or 20.

24. A microorganism comprising the vector on claim 23.

25. A vertebrate cell comprising in its genome the nucleic acid presented in any of claims 1, 6, 10, 13, 17, or 20.

26. The vertebrate cell of claim 25 that further comprises in its genome phiC31 integrase recognition sequences.

27. A transgenic organism that comprises in its genome the nucleic acid presented in any of claims 1, 6, 10, 13, 17, or 20.

28. A transgenic organism according to claim 27 that further comprises in its genome phiC31 integrase recognition sequences.

29. A method of recombining a DNA molecule containing phiC31 integrase recognition sequences in a eukaryotic cell, said method comprising contacting the cell with a phiC31 integrase according to any of claims 1, 6, 10, 13, 17, or 20, wherein the phiC31 integrase catalyzes recombination of the DNA molecule.


SEQUENCE LISTING (26 sequences)

SEQ ID NO: 1 - DNA, 1842 bp, Bacteriophage phi-C31
atgacacaaggggttgtgaccggggtggacacgtacgcgggtgcttacgaccgtcagtcgcgcgagcgcgagaattcgagcgcagcaagcccagcgacacagcgtagcgccaacgaagacaaggcggccgaccttcagcgcgaagtcgagcgcgacgggggccggttcaggttcgtcgggcatttcagcgaagcgccgggcacgtcggcgttcgggacggcggagcgcccggagttcgaacgcatcctgaacgaatgccgcgccgggcggctcaacatgatcattgtctatgacgtgtcgcgcttctcgcgcctgaaggtcatggacgcgattccgattgtctcggaattgctcgccctgggcgtgacgattgtttccactcaggaaggcgtcttccggcagggaaacgtcatggacctgattcacctgattatgcggctcgacgcgtcgcacaaagaatcttcgctgaagtcggcgaagattctcgacacgaagaaccttcagcgcgaattgggcgggtacgtcggcgggaaggcgccttacggcttcgagcttgtttcggagacgaaggagatcacgcgcaacggccgaatggtcaatgtcgtcatcaacaagcttgcgcactcgaccactccccttaccggacccttcgagttcgagcccgacgtaatccggtggtggtggcgtgagatcaagacgcacaaacaccttcccttcaagccgggcagtcaagccgccattcacccgggcagcatcacggggctttgtaagcgcatggacgctgacgccgtgccgacccggggcgagacgattgggaagaagaccgcttcaagcgcctgggacccggcaaccgttatgcgaatccttcgggacccgcgtattgcgggcttcgccgctgaggtgatctacaagaagaagccggacggcacgccgaccacgaagattgagggttaccgcattcagcgcgacccgatcacgctccggccggtcgagcttgattgcggaccgatcatcgagcccgctgagtggtatgagcttcaggcgtggttggacggcagggggcgcggcaaggggctttcccgggggcaagccattctgtccgccatggacaagctgtactgcgagtgtggcgccgtcatgacttcgaagcgcggggaagaatcgatcaaggactcttaccgctgccgtcgccggaaggtggtcgacccgtccgcacctgggcagcacgaaggcacgtgcaacgtcagcatggcggcactcgacaagttcgttgcggaacgcatcttcaacaagatcaggcacgccgaaggcgacgaagagacgttggcgcttctgtgggaagccgcccgacgcttcggcaagctcactgaggcgcctgagaagagcggcgaacgggcgaaccttgttgcggagcgcgccgacgccctgaacgcccttgaagagctgtacgaagaccgcgcggcaggcgcgtacgacggacccgttggcaggaagcacttccggaagcaacaggcagcgctgacgctccggcagcaaggggcggaagagcggcttgccgaacttgaagccgccgaagccccgaagcttccccttgaccaatggttccccgaagacgccgacgctgacccgaccggccctaagtcgtggtgggggcgcgcgtcagtagacgacaagcgcgtgttcgtcgggctcttcgtagacaagatcgttgtcacgaagtcgactacgggcagggggcagggaacgcccatcgagaagcgcgcttcgatcacgtgggcgaagccgccgaccgacgacgacgaagacgacgcccaggacggcacggaagacgtagcggcgtag

SEQ ID NO: 2 - protein, 613 aa, Bacteriophage phi-C31
MTQGVVTGVDTYAGAYDRQSRERENSSAASPATQRSANEDKAADLQREVERDGGRFRFVGHFSEAPGTSAFGTAERPEFERILNECRAGRLNMIIVYDVSRFSRLKVMDAIPIVSELLALGVTIVSTQEGVFRQGNVMDLIHLIMRLDASHKESSLKSAKILDTKNLQRELGGYVGGKAPYGFELVSETKEITRNGRMVNVVINKLAHSTTPLTGPFEFEPDVIRWWWREIKTHKHLPFKPGSQAAIHPGSITGLCKRMDADAVPTRGETIGKKTASSAWDPATVMRILRDPRIAGFAAEVIYKKKPDGTPTTKIEGYRIQRDPITLRPVELDCGPIIEPAEWYELQAWLDGRGRGKGLSRGQAILSAMDKLYCECGAVMTSKRGEESIKDSYRCRRRKVVDPSAPGQHEGTCNVSMAALDKFVAERIFNKIRHAEGDEETLALLWEAARRFGKLTEAPEKSGERANLVAERADALNALEELYEDRAAGAYDGPVGRKHFRKQQAALTLRQQGAEERLAELEAAEAPKLPLDQWFPEDADADPTGPKSWWGRASVDDKRVFVGLFVDKIVVTKSTTGRGQGTPIEKRASITWAKPPTDDDEDDAQDGTEDVAA

SEQ ID NO: 3 - DNA, 12 bp, Artificial
  Splice acceptor sequence
yyyyyyyncagg

SEQ ID NO: 4 - DNA, 12 bp, Artificial
  Splice acceptor site.
yyyyyyyntagg

SEQ ID NO: 5 - DNA, 1842 bp, C-31-Int-CO
atgacccagggcgtggtgaccggcgtggacacctacgccggagcctacgacaggcagagcagggagagggagaacagctccgccgccagccccgccacccagagatccgccaacgaggacaaggccgccgacctgcagagggaggtggagagggacggcggcaggttcaggttcgtgggccacttcagcgaggcccccggcacctccgccttcggcaccgccgagaggcccgagttcgagaggatcctgaacgagtgcagggccggcaggctgaacatgatcatcgtgtacgatgtgagcaggttcagcaggctgaaggtcatggatgccatccccatcgtgtccgagctgctggccctgggcgtgaccatcgtgagcacccaggagggcgtgttcaggcagggcaacgtgatggacctgatccacctgatcatgaggctggatgccagccacaaggagagcagcctgaagtccgccaagatcctggacaccaagaacctgcagagagagctgggcggctacgtgggcggcaaggccccctacggcttcgaactcgtgagcgagaccaaggagatcaccaggaacggcaggatggtgaacgtggtgatcaacaagctggcccacagcaccacccccctgaccggccccttcgagttcgagcccgacgtgatcaggtggtggtggagggagatcaagacccacaagcacctgcccttcaagcccggcagccaggccgccatccaccccggcagcatcaccggcctgtgcaagaggatggatgccgatgccgtgcccaccaggggcgagaccatcggcaagaaaaccgccagctccgcctgggaccccgccaccgtgatgaggatcctgagggacccccggatcgccggcttcgccgccgaggtgatctacaagaagaagcccgacggcacccccaccaccaagatcgagggctacaggatccagagggaccccatcaccctgaggcccgtggagctggactgcggccccatcatcgagcccgccgagtggtacgagctgcaggcctggctggacggcaggggcaggggcaagggcctgagcaggggccaggccatcctgtccgccatggacaagctgtactgcgagtgcggagccgtgatgaccagcaagaggggcgaggagagcatcaaggacagctacagatgcaggaggaggaaggtggtggacccctccgcccccggccagcacgagggcacctgcaacgtgagcatggccgccctggacaagttcgtggccgagaggatcttcaacaagatcaggcacgccgagggcgacgaggagaccctggccctgctgtgggaggccgccaggaggttcggcaagctgaccgaggcccccgagaagagcggcgagagggccaacctggtggccgagagggccgatgccctgaatgccctggaggagctgtacgaggacagggccgccggagcctacgacggccccgtgggcaggaagcacttcaggaagcagcaggccgccctgaccctgaggcagcagggagccgaggagaggctggccgagctggaggccgccgaggcccccaagctgcccctggaccagtggttccccgaggatgccgatgccgaccccaccggccccaagagctggtggggcagggccagcgtggacgacaagagggtgttcgtgggcctgttcgtggacaagatcgtggtgaccaagagcaccaccggcaggggccagggcacccccatcgagaagagggccagcatcacctgggccaagccccccaccgacgacgacgaggacgatgcccaggacggcaccgaggacgtggccgcctga

SEQ ID NO: 6 - DNA, 1863 bp, C-31-Int-(CNLS)
atgacccagggcgtggtgaccggcgtggacacctacgccggagcctacgacaggcagagcagggagagggagaacagctccgccgccagccccgccacccagagatccgccaacgaggacaaggccgccgacctgcagagggaggtggagagggacggcggcaggttcaggttcgtgggccacttcagcgaggcccccggcacctccgccttcggcaccgccgagaggcccgagttcgagaggatcctgaacgagtgcagggccggcaggctgaacatgatcatcgtgtacgatgtgagcaggttcagcaggctgaaggtcatggatgccatccccatcgtgtccgagctgctggccctgggcgtgaccatcgtgagcacccaggagggcgtgttcaggcagggcaacgtgatggacctgatccacctgatcatgaggctggatgccagccacaaggagagcagcctgaagtccgccaagatcctggacaccaagaacctgcagagagagctgggcggctacgtgggcggcaaggccccctacggcttcgaactcgtgagcgagaccaaggagatcaccaggaacggcaggatggtgaacgtggtgatcaacaagctggcccacagcaccacccccctgaccggccccttcgagttcgagcccgacgtgatcaggtggtggtggagggagatcaagacccacaagcacctgcccttcaagcccggcagccaggccgccatccaccccggcagcatcaccggcctgtgcaagaggatggatgccgatgccgtgcccaccaggggcgagaccatcggcaagaaaaccgccagctccgcctgggaccccgccaccgtgatgaggatcctgagggacccccggatcgccggcttcgccgccgaggtgatctacaagaagaagcccgacggcacccccaccaccaagatcgagggctacaggatccagagggaccccatcaccctgaggcccgtggagctggactgcggccccatcatcgagcccgccgagtggtacgagctgcaggcctggctggacggcaggggcaggggcaagggcctgagcaggggccaggccatcctgtccgccatggacaagctgtactgcgagtgcggagccgtgatgaccagcaagaggggcgaggagagcatcaaggacagctacagatgcaggaggaggaaggtggtggacccctccgcccccggccagcacgagggcacctgcaacgtgagcatggccgccctggacaagttcgtggccgagaggatcttcaacaagatcaggcacgccgagggcgacgaggagaccctggccctgctgtgggaggccgccaggaggttcggcaagctgaccgaggcccccgagaagagcggcgagagggccaacctggtggccgagagggccgatgccctgaatgccctggaggagctgtacgaggacagggccgccggagcctacgacggccccgtgggcaggaagcacttcaggaagcagcaggccgccctgaccctgaggcagcagggagccgaggagaggctggccgagctggaggccgccgaggcccccaagctgcccctggaccagtggttccccgaggatgccgatgccgaccccaccggccccaagagctggtggggcagggccagcgtggacgacaagagggtgttcgtgggcctgttcgtggacaagatcgtggtgaccaagagcaccaccggcaggggccagggcacccccatcgagaagagggccagcatcacctgggccaagccccccaccgacgacgacgaggacgatgcccaggacggcaccgaggacgtggccgcccccaagaagaagaggaaggtgtga

SEQ ID NO: 7 - protein, 620 aa, C-31-Int-(CNLS)
MTQGVVTGVDTYAGAYDRQSRERENSSAASPATQRSANEDKAADLQREVERDGGRFRFVGHFSEAPGTSAFGTAERPEFERILNECRAGRLNMIIVYDVSRFSRLKVMDAIPIVSELLALGVTIVSTQEGVFRQGNVMDLIHLIMRLDASHKESSLKSAKILDTKNLQRELGGYVGGKAPYGFELVSETKEITRNGRMVNVVINKLAHSTTPLTGPFEFEPDVIRWWWREIKTHKHLPFKPGSQAAIHPGSITGLCKRMDADAVPTRGETIGKKTASSAWDPATVMRILRDPRIAGFAAEVIYKKKPDGTPTTKIEGYRIQRDPITLRPVELDCGPIIEPAEWYELQAWLDGRGRGKGLSRGQAILSAMDKLYCECGAVMTSKRGEESIKDSYRCRRRKVVDPSAPGQHEGTCNVSMAALDKFVAERIFNKIRHAEGDEETLALLWEAARRFGKLTEAPEKSGERANLVAERADALNALEELYEDRAAGAYDGPVGRKHFRKQQAALTLRQQGAEERLAELEAAEAPKLPLDQWFPEDADADPTGPKSWWGRASVDDKRVFVGLFVDKIVVTKSTTGRGQGTPIEKRASITWAKPPTDDDEDDAQDGTEDVAAPKKKRKV

SEQ ID NO: 8 - DNA, 1885 bp, C-31-Int-(CNLS)-CO
gatatcgccaccatgacccagggcgtggtgaccggcgtggacacctacgccggagcctacgacaggcagagcagggagagggagaacagctccgccgccagccccgccacccagagatccgccaacgaggacaaggccgccgacctgcagagggaggtggagagggacggcggcaggttcaggttcgtgggccacttcagcgaggcccccggcacctccgccttcggcaccgccgagaggcccgagttcgagaggatcctgaacgagtgcagggccggcaggctgaacatgatcatcgtgtacgatgtgagcaggttcagcaggctgaaggtcatggatgccatccccatcgtgtccgagctgctggccctgggcgtgaccatcgtgagcacccaggagggcgtgttcaggcagggcaacgtgatggacctgatccacctgatcatgaggctggatgccagccacaaggagagcagcctgaagtccgccaagatcctggacaccaagaacctgcagagagagctgggcggctacgtgggcggcaaggccccctacggcttcgaactcgtgagcgagaccaaggagatcaccaggaacggcaggatggtgaacgtggtgatcaacaagctggcccacagcaccacccccctgaccggccccttcgagttcgagcccgacgtgatcaggtggtggtggagggagatcaagacccacaagcacctgcccttcaagcccggcagccaggccgccatccaccccggcagcatcaccggcctgtgcaagaggatggatgccgatgccgtgcccaccaggggcgagaccatcggcaagaaaaccgccagctccgcctgggaccccgccaccgtgatgaggatcctgagggacccccggatcgccggcttcgccgccgaggtgatctacaagaagaagcccgacggcacccccaccaccaagatcgagggctacaggatccagagggaccccatcaccctgaggcccgtggagctggactgcggccccatcatcgagcccgccgagtggtacgagctgcaggcctggctggacggcaggggcaggggcaagggcctgagcaggggccaggccatcctgtccgccatggacaagctgtactgcgagtgcggagccgtgatgaccagcaagaggggcgaggagagcatcaaggacagctacagatgcaggaggaggaaggtggtggacccctccgcccccggccagcacgagggcacctgcaacgtgagcatggccgccctggacaagttcgtggccgagaggatcttcaacaagatcaggcacgccgagggcgacgaggagaccctggccctgctgtgggaggccgccaggaggttcggcaagctgaccgaggcccccgagaagagcggcgagagggccaacctggtggccgagagggccgatgccctgaatgccctggaggagctgtacgaggacagggccgccggagcctacgacggccccgtgggcaggaagcacttcaggaagcagcaggccgccctgaccctgaggcagcagggagccgaggagaggctggccgagctggaggccgccgaggcccccaagctgcccctggaccagtggttccccgaggatgccgatgccgaccccaccggccccaagagctggtggggcagggccagcgtggacgacaagagggtgttcgtgggcctgttcgtggacaagatcgtggtgaccaagagcaccaccggcaggggccagggcacccccatcgagaagagggccagcatcacctgggccaagccccccaccgacgacgacgaggacgatgcccaggacggcaccgaggacgtggccgcccccaagaagaagaggaaggtgtgatgaagatatc

SEQ ID NO: 9 - DNA, 5723 bp, pCMV-C31-Int(CNLS)
ggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctagcccgggctagcttgcatgcctgcaggtttaaacagtccgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctgactctagacttaattaagcgttggggtgagtactccctctcaaaagcgggcatgacttctgcgctaagattgtcagtttccaaaaacgaggaggatttgatattcacctggcccgcggtgatgcctttgagggtggccgcgtccatctggtcagaaaagacaatctttttgttgtcaagcttgaggtgtggcaggcttgagatctggccatacacttgagtgacattgacatccactttgcctttctctccacaggtgtccactcccagggcggccgcccgatatgacacaaggggttgtgaccggggtggacacgtacgcgggtgcttacgaccgtcagtcgcgcgagcgcgagaattcgagcgcagcaagcccagcgacacagcgtagcgccaacgaagacaaggcggccgaccttcagcgcgaagtcgagcgcgacgggggccggttcaggttcgtcgggcatttcagcgaagcgccgggcacgtcggcgttcgggacggcggagcgcccggagttcgaacgcatcctgaacgaatgccgcgccgggcggctcaacatgatcattgtctatgacgtgtcgcgcttctcgcgcctgaaggtcatggacgcgattccgattgtctcggaattgctcgccctgggcgtgacgattgtttccactcaggaaggcgtcttccggcagggaaacgtcatggacctgattcacctgattatgcggctcgacgcgtcgcacaaagaatcttcgctgaagtcggcgaagattctcgacacgaagaaccttcagcgcgaattgggcgggtacgtcggcgggaaggcgccttacggcttcgagcttgtttcggagacgaaggagatcacgcgcaacggccgaatggtcaatgtcgtcatcaacaagcttgcgcactcgaccactccccttaccggacccttcgagttcgagcccgacgtaatccggtggtggtggcgtgagatcaagacgcacaaacaccttcccttcaagccgggcagtcaagccgccattcacccgggcagcatcacggggctttgtaagcgcatggacgctgacgccgtgccgacccggggcgagacgattgggaagaagaccgcttcaagcgcctgggacccggcaaccgttatgcgaatccttcgggacccgcgtattgcgggcttcgccgctgaggtgatctacaagaagaagccggacggcacgccgaccacgaagattgagggttaccgcattcagcgcgacccgatcacgctccggccggtcgagcttgattgcggaccgatcatcgagcccgctgagtggtatgagcttcaggcgtggttggacggcagggggcgcggcaaggggctttcccgggggcaagccattctgtccgccatggacaagctgtactgcgagtgtggcgccgtcatgacttcgaagcgcggggaagaatcgatcaaggactcttaccgctgccgtcgccggaaggtggtcgacccgtccgcacctgggcagcacgaaggcacgtgcaacgtcagcatggcggcactcgacaagttcgttgcggaacgcatcttcaacaagatcaggcacgccgaaggcgacgaagagacgttggcgcttctgtgggaagccgcccgacgcttcggcaagctcactgaggcgcctgagaagagcggcgaacgggcgaaccttgttgcggagcgcgccgacgccctgaacgcccttgaagagctgtacgaagaccgcgcggcaggcgcgtacgacggacccgttggcaggaagcacttccggaagcaacaggcagcgctgacgctccggcagcaaggggcggaagagcggcttgccgaacttgaagccgccgaagccccgaagcttccccttgaccaatggttccccgaagacgccgacgctgacccgaccggccctaagtcgtggtgggggcgcgcgtcagtagacgacaagcgcgtgttcgtcgggctcttcgtagacaagatcgttgtcacgaagtcgactacgggcagggggcagggaacgcccatcgagaagcgcgcttcgatcacgtgggcgaagccgccgaccgacgacgacgaagacgacgcccaggacggcacggaagacgtagcggcgcctaagaagaagaggaaggtttagactctcgagatccaggcgcggatcaataaaagatcattattttcaatagatctgtgtgttggttttttgtgtgccttgggggagggggaggccagaatgaggcgcggccaagggggagggggaggccagaatgaccttgggggagggggaggccagaatgaccttgggggagggggaggccagaatgaggcgcgcccccgggtaccgagctcgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagct

SEQ ID NO: 10 - DNA, 5741 bp, pCMV-C31-Ins(CNLS)-CO
ggccagcgtggacgacaagagggtgttcgtgggcctgttcgtggacaagatcgtggtgaccaagagcaccaccggcaggggccagggcacccccatcgagaagagggccagcatcacctgggccaagccccccaccgacgacgacgaggacgatgcccaggacggcaccgaggacgtggccgcccccaagaagaagaggaaggtgtgatgaagatggccgcgtcgacctcgagatccaggcgcggatcaataaaagatcattattttcaatagatctgtgtgttggttttttgtgtgccttgggggagggggaggccagaatgaggcgcggccaagggggagggggaggccagaatgaccttgggggagggggaggccagaatgaccttgggggagggggaggccagaatgaggcgcgcccccgggtaccgagctcgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctagcccgggctagcttgcatgcctgcaggtttaaacagtccgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctgactctagacttaattaagcgttggggtgagtactccctctcaaaagcgggcatgacttctgcgctaagattgtcagtttccaaaaacgaggaggatttgatattcacctggcccgcggtgatgcctttgagggtggccgcgtccatctggtcagaaaagacaatctttttgttgtcaagcttgaggtgtggcaggcttgagatctggccatacacttgagtgacattgacatccactttgcctttctctccacaggtgtccactcccagggcggccatcgccaccatgacccagggcgtggtgaccggcgtggacacctacgccggagcctacgacaggcagagcagggagagggagaacagctccgccgccagccccgccacccagagatccgccaacgaggacaaggccgccgacctgcagagggaggtggagagggacggcggcaggttcaggttcgtgggccacttcagcgaggcccccggcacctccgccttcggcaccgccgagaggcccgagttcgagaggatcctgaacgagtgcagggccggcaggctgaacatgatcatcgtgtacgatgtgagcaggttcagcaggctgaaggtcatggatgccatccccatcgtgtccgagctgctggccctgggcgtgaccatcgtgagcacccaggagggcgtgttcaggcagggcaacgtgatggacctgatccacctgatcatgaggctggatgccagccacaaggagagcagcctgaagtccgccaagatcctggacaccaagaacctgcagagagagctgggcggctacgtgggcggcaaggccccctacggcttcgaactcgtgagcgagaccaaggagatcaccaggaacggcaggatggtgaacgtggtgatcaacaagctggcccacagcaccacccccctgaccggccccttcgagttcgagcccgacgtgatcaggtggtggtggagggagatcaagacccacaagcacctgcccttcaagcccggcagccaggccgccatccaccccggcagcatcaccggcctgtgcaagaggatggatgccgatgccgtgcccaccaggggcgagaccatcggcaagaaaaccgccagctccgcctgggaccccgccaccgtgatgaggatcctgagggacccccggatcgccggcttcgccgccgaggtgatctacaagaagaagcccgacggcacccccaccaccaagatcgagggctacaggatccagagggaccccatcaccctgaggcccgtggagctggactgcggccccatcatcgagcccgccgagtggtacgagctgcaggcctggctggacggcaggggcaggggcaagggcctgagcaggggccaggccatcctgtccgccatggacaagctgtactgcgagtgcggagccgtgatgaccagcaagaggggcgaggagagcatcaaggacagctacagatgcaggaggaggaaggtggtggacccctccgcccccggccagcacgagggcacctgcaacgtgagcatggccgccctggacaagttcgtggccgagaggatcttcaacaagatcaggcacgccgagggcgacgaggagaccctggccctgctgtgggaggccgccaggaggttcggcaagctgaccgaggcccccgagaagagcggcgagagggccaacctggtggccgagagggccgatgccctgaatgccctggaggagctgtacgaggacagggccgccggagcctacgacggccccgtgggcaggaagcacttcaggaagcagcaggccgccctgaccctgaggcagcagggagccgaggagaggctggccgagctggaggccgccgaggcccccaagctgcccctggaccagtggttccccgaggatgccgatgccgaccccaccggccccaagagctggtggggcag

SEQ ID NO: 11 - DNA, 7438 bp, pRK64
cgtcatcaccgaaacgcgcgaggcagctgtggaatgtgtgtcagttagggtgtggaaagtccccaggctccccagcaggcagaagtatgcaaagcatgcatctcaattagtcagcaaccaggctccccagcaggcagaagtgtgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctaggaacagtcgacgacactgcagagacctacttcactaacaaccggtacagttcgtggaccagatgggtgaggtggagtacgcgcccggggagcccaagggcacgccctggcacccgcaccgcggcttcgagaccgtcacgaataacttcgtatagcatacattatacgaagttataagcttgcatgcctgcaggtcggccgccacgaccggccggccggtgccgccaccatcccctgacccacgcccctgacccctcacaaggagacgaccttccatgaccgagtacaagcccacggtgcgcctcgccacccgcgacgacgtcccccgggccgtacgcaccctcgccgccgcgttcgccgactaccccgccacgcgccacaccgtcgacccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgagtgcccgaaggaccgcgcgacctggtgcatgacccgcaagcccggtgcctgacgcccgccccacgacccgcagcgcccgaccgaaaggagcgcacgaccccatggctccgaccgaagccgacccgggcggccccgccgaccccgcacccgcccccgaggcccaccgactctagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatccgtgtcatgtcggcgaccctacgcccccaactgagagaactcaaaggttaccccagttggggcactactcccgaaaaccgcttctggatccataacttcgtatagcatacattatacgaagttataccgggccaccatggtcgcgagtagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtaacctatcccattacggtcaatccgccgtttgttcccacggagaatccgacgggttgttactcgctcacatttaatgttgatgaaagctggctacaggaaggccagacgcgaattatttttgatggcgttaactcggcgtttcatctgtggtgcaacgggcgctgggtcggttacggccaggacagtcgtttgccgtctgaatttgacctgagcgcatttttacgcgccggagaaaaccgcctcgcggtgatggtgctgcgttggagtgacggcagttatctggaagatcaggatatgtggcggatgagcggcattttccgtgacgtctcgttgctgcataaaccgactacacaaatcagcgatttccatgttgccactcgctttaatgatgatttcagccgcgctgtactggaggctgaagttcagatgtgcggcgagttgcgtgactacctacgggtaacagtttctttatggcagggtgaaacgcaggtcgccagcggcaccgcgcctttcggcggtgaaattatcgatgagcgtggtggttatgccgatcgcgtcacactacgtctgaacgtcgaaaacccgaaactgtggagcgccgaaatcccgaatctctatcgtgcggtggttgaactgcacaccgccgacggcacgctgattgaagcagaagcctgcgatgtcggtttccgcgaggtgcggattgaaaatggtctgctgctgctgaacggcaagccgttgctgattcgaggcgttaaccgtcacgagcatcatcctctgcatggtcaggtcatggatgagcagacgatggtgcaggatatcctgctgatgaagcagaacaactttaacgccgtgcgctgttcgcattatccgaaccatccgctgtggtacacgctgtgcgaccgctacggcctgtatgtggtggatgaagccaatattgaaacccacggcatggtgccaatgaatcgtctgaccgatgatccgcgctggctaccggcgatgagcgaacgcgtaacgcgaatggtgcagcgcgatcgtaatcacccgagtgtgatcatctggtcgctggggaatgaatcaggccacggcgctaatcacgacgcgctgtatcgctggatcaaatctgtcgatccttcccgcccggtgcagtatgaaggcggcggagccgacaccacggccaccgatattatttgcccgatgtacgcgcgcgtggatgaagaccagcccttcccggctgtgccgaaatggtccatcaaaaaatggctttcgctacctggagagacgcgcccgctgatcctttgcgaatacgcccacgcgatgggtaacagtcttggcggtttcgctaaatactggcaggcgtttcgtcagtatccccgtttacagggcggcttcgtctgggactgggtggatcagtcgctgattaaatatgatgaaaacggcaacccgtggtcggcttacggcggtgattttggcgatacgccgaacgatcgccagttctgtatgaacggtctggtctttgccgaccgcacgccgcatccagcgctgacggaagcaaaacaccagcagcagtttttccagttccgtttatccgggcaaaccatcgaagtgaccagcgaatacctgttccgtcatagcgataacgagctcctgcactggatggtggcgctggatggtaagccgctggcaagcggtgaagtgcctctggatgtcgctccacaaggtaaacagttgattgaactgcctgaactaccgcagccggagagcgccgggcaactctggctcacagtacgcgtagtgcaaccgaacgcgaccgcatggtcagaagccgggcacatcagcgcctggcagcagtggcgtctggcggaaaacctcagtgtgacgctccccgccgcgtcccacgccatcccgcatctgaccaccagcgaaatggatttttgcatcgagctgggtaataagcgttggcaatttaaccgccagtcaggctttctttcacagatgtggattggcgataaaaaacaactgctgacgccgctgcgcgatcagttcacccgtgcaccgctggataacgacattggcgtaagtgaagcgacccgcattgaccctaacgcctgggtcgaacgctggaaggcggcgggccattaccaggccgaagcagcgttgttgcagtgcacggcagatacacttgctgatgcggtgctgattacgaccgctcacgcgtggcagcatcaggggaaaaccttatttatcagccggaaaacctaccggattgatggtagtggtcaaatggcgattaccgttgatgttgaagtggcgagcgatacaccgcatccggcgcggattggcctgaactgccagctggcgcaggtagcagagcgggtaaactggctcggattagggccgcaagaaaactatcccgaccgccttactgccgcctgttttgaccgctgggatctgccattgtcagacatgtataccccgtacgtcttcccgagcgaaaacggtctgcgctgcgggacgcgcgaattgaattatggcccacaccagtggcgcggcgacttccagttcaacatcagccgctacagtcaacagcaactgatggaaaccagccatcgccatctgctgcacgcggaagaaggcacatggctgaatatcgacggtttccatatggggattggtggcgacgactcctggagcccgtcagtatcggcggaattccagctgagcgccggtcgctaccattaccagttggtctggtgtcaaaaataataataaccgggcaggggggatctttgtgaaggaaccttacttctgtggtgtgacataattggacaaactacctacagagatttaaagctctaaggtaaatataaaatttttaagtgtataatgtgttaaactactgattctaattgtttgtgtattttagattccaacctatggaactgatgaatgggagcagtggtggaatgccagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatctgcggccgcagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctggcgcg

SEQ ID NO: 12 - DNA, 12538 bp, pROSA-SA-C31-Int(CNLS)
tttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctgtcgagatctagatatcgatggccatagagttacgctagggataacagggtaatatagccgcggcaggccctccgagcgtggtggagccgttctgtgagacagccgggtacgagtcgtgacgctggaaggggcaagcgggtggtgggcaggaatgcggtccgccctgcagcaaccggagggggagggagaagggagcggaaaagtctccaccggacgcggccatggctcgggggggggggggcagcggaggagcgcttccggccgacgtctcgtcgctgattggcttcttttcctcccgccgtgtgtgaaaacacaaatggcgtgttttggttggcgtaaggcgcctgtcagttaacggcagccggagtgcgcagccgccggcagcctcgctctgcccactgggtggggcgggaggtaggtggggtgaggcgagctggacgtgcgggcgcggtcggcctctggcggggcgggggaggggagggagggtcagcgaaagtagctcgcgcgcgagcggccgcccaccctccccttcctctgggggagtcgttttacccgccgccggccgggcctcgtcgtctgattggctctcggggcccagaaaactggcccttgccattggctcgtgttcgtgcaagttgagtccatccgccggccagcgggggcggcgaggaggcgctcccaggttccggccctcccctcggccccgcgccgcagagtctggccgcgcgcccctgcgcaacgtggcaggaagcgcgcgctgggggcggggacgggcagtagggctgagcggctgcggggcgggtgcaagcacgtttccgacttgagttgcctcaagaggggcgtgctgagccagacctccatcgcgcactccggggagtggagggaaggagcgagggctcagttgggctgttttggaggcaggaagcacttgctctcccaaagtcgctctgagttgttatcagtaagggagctgcagtggagtaggcggggagaaggccgcacccttctccggaggggggaggggagtgttgcaatacctttctgggagttctctgctgcctcctggcttctgaggaccgccctgggcctgggagaatcccttccccctcttccctcgtgatctgcaactccagtctttctaggtaaccgatatccctgcaggggtgacctgcacgtctagggcgcagtagtccagggtttccttgatgatgtcatacttatcctgtcccttttttttccacagctcgcggttgaggacaaactcttcgcggtctttccagtactcctgcaggtgactgactgagtcgacttaattaaggccatagcggccatttggccgcccgatatgacacaaggggttgtgaccggggtggacacgtacgcgggtgcttacgaccgtcagtcgcgcgagcgcgagaattcgagcgcagcaagcccagcgacacagcgtagcgccaacgaagacaaggcggccgaccttcagcgcgaagtcgagcgcgacgggggccggttcaggttcgtcgggcatttcagcgaagcgccgggcacgtcggcgttcgggacggcggagcgcccggagttcgaacgcatcctgaacgaatgccgcgccgggcggctcaacatgatcattgtctatgacgtgtcgcgcttctcgcgcctgaaggtcatggacgcgattccgattgtctcggaattgctcgccctgggcgtgacgattgtttccactcaggaaggcgtcttccggcagggaaacgtcatggacctgattcacctgattatgcggctcgacgcgtcgcacaaagaatcttcgctgaagtcggcgaagattctcgacacgaagaaccttcagcgcgaattgggcgggtacgtcggcgggaaggcgccttacggcttcgagcttgtttcggagacgaaggagatcacgcgcaacggccgaatggtcaatgtcgtcatcaacaagcttgcgcactcgaccactccccttaccggacccttcgagttcgagcccgacgtaatccggtggtggtggcgtgagatcaagacgcacaaacaccttcccttcaagccgggcagtcaagccgccattcacccgggcagcatcacggggctttgtaagcgcatggacgctgacgccgtgccgacccggggcgagacgattgggaagaagaccgcttcaagcgcctgggacccggcaaccgttatgcgaatccttcgggacccgcgtattgcgggcttcgccgctgaggtgatctacaagaagaagccggacggcacgccgaccacgaagattgagggttaccgcattcagcgcgacccgatcacgctccggccggtcgagcttgattgcggaccgatcatcgagcccgctgagtggtatgagcttcaggcgtggttggacggcagggggcgcggcaaggggctttcccgggggcaagccattctgtccgccatggacaagctgtactgcgagtgtggcgccgtcatgacttcgaagcgcggggaagaatcgatcaaggactcttaccgctgccgtcgccggaaggtggtcgacccgtccgcacctgggcagcacgaaggcacgtgcaacgtcagcatggcggcactcgacaagttcgttgcggaacgcatcttcaacaagatcaggcacgccgaaggcgacgaagagacgttggcgcttctgtgggaagccgcccgacgcttcggcaagctcactgaggcgcctgagaagagcggcgaacgggcgaaccttgttgcggagcgcgccgacgccctgaacgcccttgaagagctgtacgaagaccgcgcggcaggcgcgtacgacggacccgttggcaggaagcacttccggaagcaacaggcagcgctgacgctccggcagcaaggggcggaagagcggcttgccgaacttgaagccgccgaagccccgaagcttccccttgaccaatggttccccgaagacgccgacgctgacccgaccggccctaagtcgtggtgggggcgcgcgtcagtagacgacaagcgcgtgttcgtcgggctcttcgtagacaagatcgttgtcacgaagtcgactacgggcagggggcagggaacgcccatcgagaagcgcgcttcgatcacgtgggcgaagccgccgaccgacgacgacgaagacgacgcccaggacggcacggaagacgtagcggcgcctaagaagaagaggaaggtttagactctcgagatccaggcgcggatcaataaaagatcattattttcaatagatctgtgtgttggttttttgtgtgccttgggggagggggaggccagaatgaggcgcggccaagggggagggggaggccagaatgaccttgggggagggggaggccagaatgaccttgggggagggggaggccagaatgaggcgcgccggtaaccgaagttcctatactttctagagaataggaacttcggaataggaacttcttaggtcaattctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacctgcagccaatatgggatcggccattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaggggatcgatccgctgtaagtctgcagaaattgatgatctattaaacaataaagatgtccactaaaatggaagtttttcctgtcatactttgttaagaagggtgagaacagagtacctacattttgaatggaaggattggagctacgggggtgggggtggggtgggattagataaatgcctgctctttactgaaggctctttactattgctttatgataatgtttcatagttggatatcataatttaaacaagcaaaaccaaattaagggccagctcattcctcccactcatgatctatagatctatagatctctcgtgggatcattgtttttctcttgattcccactttgtggttctaagtactgtggtttccaaatgtgtcagtttcatagcctgaagaacgagatcagcagcctctgttccacatacacttcattctcagtattgttttgccaagttctaattccatcagaagctgactctagatcccgcgccgaagttcctatactttctagagaataggaacttcggaataggaacttcaagcttaagcgctagaagatgggcgggagtcttctgggcaggcttaaaggctaacctggtgtgtgggcgttgtcctgcaggggaattgaacaggtgtaaaattggagggacaagacttcccacagattttcggttttgtcgggaagttttttaataggggcaaataaggaaaatgggaggataggtagtcatctggggttttatgcagcaaaactacaggttattattgcttgtgatccgcctcggagtattttccatcgaggtagattaaagacatgctcacccgagttttatactctcctgcttgagatccttactacagtatgaaattacagtgtcgcgagttagactatgtaagcagaattttaatcatttttaaagagcccagtacttcatatccatttctcccgctccttctgcagccttatcaaaaggtattttagaacactcattttagccccattttcatttattatactggcttatccaacccctagacagagcattggcattttccctttcctgatcttagaagtctgatgactcatgaaaccagacagattagttacatacaccacaaatcgaggctgtagctggggcctcaacactgcagttcttttataactccttagtacactttttgttgatcctttgccttgatccttaattttcagtgtctatcacctctcccgtcagtggtgttccacatttgggcctattctcagtccagggagttttacaacaatagatgtattgagaatccaacctaaagcttaactttccactcccatgaatgcctctctcctttttctccatttataaactgagctattaaccattaatggttccaggtggatgtctcctccccatattacctgatgtatcttacatattgccaggctgatattttaagacattaaaaggtatatttcattattgagccacatggtattgattactgcttactaaaattttgtcattgtacacatctgtaaaaggtggttccttttggaatgcaaagttcaggtgtttgttgtctttcctgacctaaggtcttgtgagcttgtattttttctatttaagcagtgctttctcttggactggcttgactcatggcattctacacgttattgctggtctaaatgtgattttgccaagcttcttcaggacctataattttgcttgacttgtagccaaacacaagtaaaatgattaagcaacaaatgtatttgtgaagcttggtttttaggttgttgtgttgtgtgtgcttgtgctctataataatactatccaggggctggagaggtggctcggagttcaagagcacagactgctcttccagaagtcctgagttcaattcccagcaaccacatggtggctcacaaccatctgtaatgggatctgatgccctcttctggtgtgtctgaagaccacaagtgtattcacattaaataaataaatcctccttcttcttcttttttttttttttaaagagaatactgtctccagtagaatttactgaagtaatgaaatactttgtgtttgttccaatatggtagccaataatcaaattactctttaagcactggaaatgttaccaaggaactaatttttatttgaagtgtaactgtggacagaggagccataactgcagacttgtgggatacagaagaccaatgcagactttaatgtcttttctcttacactaagcaataaagaaataaaaattgaacttctagtatcctatttgtttaaactgctagctttacttaacttttgtgcttcatctatacaaagctgaaagctaagtctgcagccattactaaacatgaaagcaagtaatgataattttggatttcaaaaatgtagggccagagtttagccagccagtggtggtgcttgcctttatgcctttaatcccagcactctggaggcagagacaggcagatctctgagtttgagcccagcctggtctacacatcaagttctatctaggatagccaggaatacacacagaaaccctgttggggaggggggctctgagatttcataaaattataattgaagcattccctaatgagccactatggatgtggctaaatccgtctacctttctgatgagatttgggtattattttttctgtctctgctgttggttgggtcttttgacactgtgggctttctttaaagcctccttcctgccatgtggtctcttgtttgctactaacttcccatggcttaaatggcatggctttttgccttctaagggcagctgctgagatttgcagcctgatttccagggtggggttgggaaatctttcaaacactaaaattgtcctttaattttttttttaaaaaatgggttatataataaacctcataaaatagttatgaggagtgaggtggactaatattaaatgagtccctcccctataaaagagctattaaggctttttgtcttatacttaactttttttttaaatgtggtatctttagaaccaagggtcttagagttttagtatacagaaactgttgcatcgcttaatcagattttctagtttcaaatccagagaatccaaattcttcacagccaaagtcaaattaagaatttctgacttttaatgttaatttgcttactgtgaatataaaaatgatagcttttcctgaggcagggtctcactatgtatctctgcctgatctgcaacaagatatgtagactaaagttctgcctgcttttgtctcctgaatactaaggttaaaatgtagtaatacttttggaacttgcaggtcagattcttttataggggacacactaagggagcttgggtgatagttggtaaaatgtgtttcaagtgatgaaaacttgaattattatcaccgcaacctactttttaaaaaaaaaagccaggcctgttagagcatgcttaagggatccctaggacttgctgagcacacaagagtagttacttggcaggctcctggtgagagcatatttcaaaaaacaaggcagacaaccaagaaactacagttaaggttacctgtctttaaaccatctgcatatacacagggatattaaaatattccaaataatatttcattcaagttttcccccatcaaattgggacatggatttctccggtgaataggcagagttggaaactaaacaaatgttggttttgtgatttgtgaaattgttttcaagtgatagttaaagcccatgagatacagaacaaagctgctatttcgaggtctcttggtttatactcagaagcacttctttgggtttccctgcactatcctgatcatgtgctaggcctaccttaggctgattgttgttcaaataaacttaagtttcctgtcaggtgatgtcatatgatttcatatatcaaggcaaaacatgttatatatgttaaacatttgtacttaatgtgaaagttaggtctttgtgggtttgatttttaattttcaaaacctgagctaaataagtcatttttacatgtcttacatttggtggaattgtataattgtggtttgcaggcaagactctctgacctagtaaccctacctatagagcactttgctgggtcacaagtctaggagtcaagcatttcaccttgaagttgagacgttttgttagtgtatactagtttatatgttggaggacatgtttatccagaagatattcaggactatttttgactgggctaaggaattgattctgattagcactgttagtgagcattgagtggcctttaggcttgaattggagtcacttgtatatctcaaataatgctggccttttttaaaaagcccttgttctttatcaccctgttttctacataatttttgttcaaagaaatacttgtttggatctccttttgacaacaatagcatgttttcaagccatattttttttcctttttttttttttttttggtttttcgagacagggtttctctgtatagccctggctgtcctggaactcactttgtagaccaggctggcctcgaactcagaaatccgcctgcctctgcctcctgagtgccgggattaaaggcgtgcaccaccacgcctggctaagttggatattttgttatataactataaccaatactaactccactgggtggatttttaattcagtcagtagtcttaagtggtctttattggcccttcattaaaatctactgttcactctaacagaggctgttggtactagtggcacttaagcaacttcctacggatatactagcagattaagggtcagggatagaaactagtctagcgttttgtatacctaccagctttatactaccttgttctgatagaaatatttcaggacatctagcacccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcc

SEQ ID NO: 13 - DNA, 12645 bp, pROSA-SA-C31-Int(CNLS)-CO
tttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctgtcgagatctagatatcgatggccatagagttacgctagggataacagggtaatatagccgcggcaggccctccgagcgtggtggagccgttctgtgagacagccgggtacgagtcgtgacgctggaaggggcaagcgggtggtgggcaggaatgcggtccgccctgcagcaaccggagggggagggagaagggagcggaaaagtctccaccggacgcggccatggctcgggggggggggggcagcggaggagcgcttccggccgacgtctcgtcgctgattggcttcttttcctcccgccgtgtgtgaaaacacaaatggcgtgttttggttggcgtaaggcgcctgtcagttaacggcagccggagtgcgcagccgccggcagcctcgctctgcccactgggtggggcgggaggtaggtggggtgaggcgagctggacgtgcgggcgcggtcggcctctggcggggcgggggaggggagggagggtcagcgaaagtagctcgcgcgcgagcggccgcccaccctccccttcctctgggggagtcgttttacccgccgccggccgggcctcgtcgtctgattggctctcggggcccagaaaactggcccttgccattggctcgtgttcgtgcaagttgagtccatccgccggccagcgggggcggcgaggaggcgctcccaggttccggccctcccctcggccccgcgccgcagagtctggccgcgcgcccctgcgcaacgtggcaggaagcgcgcgctgggggcggggacgggcagtagggctgagcggctgcggggcgggtgcaagcacgtttccgacttgagttgcctcaagaggggcgtgctgagccagacctccatcgcgcactccggggagtggagggaaggagcgagggctcagttgggctgttttggaggcaggaagcacttgctctcccaaagtcgctctgagttgttatcagtaagggagctgcagtggagtaggcggggagaaggccgcacccttctccggaggggggaggggagtgttgcaatacctttctgggagttctctgctgcctcctggcttctgaggaccgccctgggcctgggagaatcccttccccctcttccctcgtgatctgcaactccagtctttctaggtaaccgatatccctgcaggggtgacctgcacgtctagggcgcagtagtccagggtttccttgatgatgtcatacttatcctgtcccttttttttccacagctcgcggttgaggacaaactcttcgcggtctttccagtactcctgcaggtgactgactgagtcgacttaattaaggccatagcggccatttaaatagttacgctagggataacagggtaatatagttaattaatctagaactagtggatcccccgggctgcaggaattcgatatcgccaccatgacccagggcgtggtgaccggcgtggacacctacgccggagcctacgacaggcagagcagggagagggagaacagctccgccgccagccccgccacccagagatccgccaacgaggacaaggccgccgacctgcagagggaggtggagagggacggcggcaggttcaggttcgtgggccacttcagcgaggcccccggcacctccgccttcggcaccgccgagaggcccgagttcgagaggatcctgaacgagtgcagggccggcaggctgaacatgatcatcgtgtacgatgtgagcaggttcagcaggctgaaggtcatggatgccatccccatcgtgtccgagctgctggccctgggcgtgaccatcgtgagcacccaggagggcgtgttcaggcagggcaacgtgatggacctgatccacctgatcatgaggctggatgccagccacaaggagagcagcctgaagtccgccaagatcctggacaccaagaacctgcagagagagctgggcggctacgtgggcggcaaggccccctacggcttcgaactcgtgagcgagaccaaggagatcaccaggaacggcaggatggtgaacgtggtgatcaacaagctggcccacagcaccacccccctgaccggccccttcgagttcgagcccgacgtgatcaggtggtggtggagggagatcaagacccacaagcacctgcccttcaagcccggcagccaggccgccatccaccccggcagcatcaccggcctgtgcaagaggatggatgccgatgccgtgcccaccaggggcgagaccatcggcaagaaaaccgccagctccgcctgggaccccgccaccgtgatgaggatcctgagggacccccggatcgccggcttcgccgccgaggtgatctacaagaagaagcccgacggcacccccaccaccaagatcgagggctacaggatccagagggaccccatcaccctgaggcccgtggagctggactgcggccccatcatcgagcccgccgagtggtacgagctgcaggcctggctggacggcaggggcaggggcaagggcctgagcaggggccaggccatcctgtccgccatggacaagctgtactgcgagtgcggagccgtgatgaccagcaagaggggcgaggagagcatcaaggacagctacagatgcaggaggaggaaggtggtggacccctccgcccccggccagcacgagggcacctgcaacgtgagcatggccgccctggacaagttcgtggccgagaggatcttcaacaagatcaggcacgccgagggcgacgaggagaccctggccctgctgtgggaggccgccaggaggttcggcaagctgaccgaggcccccgagaagagcggcgagagggccaacctggtggccgagagggccgatgccctgaatgccctggaggagctgtacgaggacagggccgccggagcctacgacggccccgtgggcaggaagcacttcaggaagcagcaggccgccctgaccctgaggcagcagggagccgaggagaggctggccgagctggaggccgccgaggcccccaagctgcccctggaccagtggttccccgaggatgccgatgccgaccccaccggccccaagagctggtggggcagggccagcgtggacgacaagagggtgttcgtgggcctgttcgtggacaagatcgtggtgaccaagagcaccaccggcaggggccagggcacccccatcgagaagagggccagcatcacctgggccaagccccccaccgacgacgacgaggacgatgcccaggacggcaccgaggacgtggccgcccccaagaagaagaggaaggtgtgatgaagatatcaagcttatcgataccgtcgacctcgagatccaggcgcggatcaataaaagatcattattttcaatagatctgtgtgttggttttttgtgtgccttgggggagggggaggccagaatgaggcgcggccaagggggagggggaggccagaatgaccttgggggagggggaggccagaatgaccttgggggagggggaggccagaatgaggcgcgccggtaaccgaagttcctatactttctagagaataggaacttcggaataggaacttcttaggtcaattctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacctgcagccaatatgggatcggccattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaggggatcgatccgctgtaagtctgcagaaattgatgatctattaaacaataaagatgtccactaaaatggaagtttttcctgtcatactttgttaagaagggtgagaacagagtacctacattttgaatggaaggattggagctacgggggtgggggtggggtgggattagataaatgcctgctctttactgaaggctctttactattgctttatgataatgtttcatagttggatatcataatttaaacaagcaaaaccaaattaagggccagctcattcctcccactcatgatctatagatctatagatctctcgtgggatcattgtttttctcttgattcccactttgtggttctaagtactgtggtttccaaatgtgtcagtttcatagcctgaagaacgagatcagcagcctctgttccacatacacttcattctcagtattgttttgccaagttctaattccatcagaagctgactctagatcccgcgccgaagttcctatactttctagagaataggaacttcggaataggaacttcaagcttaagcgctagaagatgggcgggagtcttctgggcaggcttaaaggctaacctggtgtgtgggcgttgtcctgcaggggaattgaacaggtgtaaaattggagggacaagacttcccacagattttcggttttgtcgggaagttttttaataggggcaaataaggaaaatgggaggataggtagtcatctggggttttatgcagcaaaactacaggttattattgcttgtgatccgcctcggagtattttccatcgaggtagattaaagacatgctcacccgagttttatactctcctgcttgagatccttactacagtatgaaattacagtgtcgcgagttagactatgtaagcagaattttaatcatttttaaagagcccagtacttcatatccatttctcccgctccttctgcagccttatcaaaaggtattttagaacactcattttagccccattttcatttattatactggcttatccaacccctagacagagcattggcattttccctttcctgatcttagaagtctgatgactcatgaaaccagacagattagttacatacaccacaaatcgaggctgtagctggggcctcaacactgcagttcttttataactccttagtacactttttgttgatcctttgccttgatccttaattttcagtgtctatcacctctcccgtcagtggtgttccacatttgggcctattctcagtccagggagttttacaacaatagatgtattgagaatccaacctaaagcttaactttccactcccatgaatgcctctctcctttttctccatttataaactgagctattaaccattaatggttccaggtggatgtctcctccccatattacctgatgtatcttacatattgccaggctgatattttaagacattaaaaggtatatttcattattgagccacatggtattgattactgcttactaaaattttgtcattgtacacatctgtaaaaggtggttccttttggaatgcaaagttcaggtgtttgttgtctttcctgacctaaggtcttgtgagcttgtattttttctatttaagcagtgctttctcttggactggcttgactcatggcattctacacgttattgctggtctaaatgtgattttgccaagcttcttcaggacctataattttgcttgacttgtagccaaacacaagtaaaatgattaagcaacaaatgtatttgtgaagcttggtttttaggttgttgtgttgtgtgtgcttgtgctctataataatactatccaggggctggagaggtggctcggagttcaagagcacagactgctcttccagaagtcctgagttcaattcccagcaaccacatggtggctcacaaccatctgtaatgggatctgatgccctcttctggtgtgtctgaagaccacaagtgtattcacattaaataaataaatcctccttcttcttcttttttttttttttaaagagaatactgtctccagtagaatttactgaagtaatgaaatactttgtgtttgttccaatatggtagccaataatcaaattactctttaagcactggaaatgttaccaaggaactaatttttatttgaagtgtaactgtggacagaggagccataactgcagacttgtgggatacagaagaccaatgcagactttaatgtcttttctcttacactaagcaataaagaaataaaaattgaacttctagtatcctatttgtttaaactgctagctttacttaacttttgtgcttcatctatacaaagctgaaagctaagtctgcagccattactaaacatgaaagcaagtaatgataattttggatttcaaaaatgtagggccagagtttagccagccagtggtggtgcttgcctttatgcctttaatcccagcactctggaggcagagacaggcagatctctgagtttgagcccagcctggtctacacatcaagttctatctaggatagccaggaatacacacagaaaccctgttggggaggggggctctgagatttcataaaattataattgaagcattccctaatgagccactatggatgtggctaaatccgtctacctttctgatgagatttgggtattattttttctgtctctgctgttggttgggtcttttgacactgtgggctttctttaaagcctccttcctgccatgtggtctcttgtttgctactaacttcccatggcttaaatggcatggctttttgccttctaagggcagctgctgagatttgcagcctgatttccagggtggggttgggaaatctttcaaacactaaaattgtcctttaattttttttttaaaaaatgggttatataataaacctcataaaatagttatgaggagtgaggtggactaatattaaatgagtccctcccctataaaagagctattaaggctttttgtcttatacttaactttttttttaaatgtggtatctttagaaccaagggtcttagagttttagtatacagaaactgttgcatcgcttaatcagattttctagtttcaaatccagagaatccaaattcttcacagccaaagtcaaattaagaatttctgacttttaatgttaatttgcttactgtgaatataaaaatgatagcttttcctgaggcagggtctcactatgtatctctgcctgatctgcaacaagatatgtagactaaagttctgcctgcttttgtctcctgaatactaaggttaaaatgtagtaatacttttggaacttgcaggtcagattcttttataggggacacactaagggagcttgggtgatagttggtaaaatgtgtttcaagtgatgaaaacttgaattattatcaccgcaacctactttttaaaaaaaaaagccaggcctgttagagcatgcttaagggatccctaggacttgctgagcacacaagagtagttacttggcaggctcctggtgagagcatatttcaaaaaacaaggcagacaaccaagaaactacagttaaggttacctgtctttaaaccatctgcatatacacagggatattaaaatattccaaataatatttcattcaagttttcccccatcaaattgggacatggatttctccggtgaataggcagagttggaaactaaacaaatgttggttttgtgatttgtgaaattgttttcaagtgatagttaaagcccatgagatacagaacaaagctgctatttcgaggtctcttggtttatactcagaagcacttctttgggtttccctgcactatcctgatcatgtgctaggcctaccttaggctgattgttgttcaaataaacttaagtttcctgtcaggtgatgtcatatgatttcatatatcaaggcaaaacatgttatatatgttaaacatttgtacttaatgtgaaagttaggtctttgtgggtttgatttttaattttcaaaacctgagctaaataagtcatttttacatgtcttacatttggtggaattgtataattgtggtttgcaggcaagactctctgacctagtaaccctacctatagagcactttgctgggtcacaagtctaggagtcaagcatttcaccttgaagttgagacgttttgttagtgtatactagtttatatgttggaggacatgtttatccagaagatattcaggactatttttgactgggctaaggaattgattctgattagcactgttagtgagcattgagtggcctttaggcttgaattggagtcacttgtatatctcaaataatgctggccttttttaaaaagcccttgttctttatcaccctgttttctacataatttttgttcaaagaaatacttgtttggatctccttttgacaacaatagcatgttttcaagccatattttttttcctttttttttttttttttggtttttcgagacagggtttctctgtatagccctggctgtcctggaactcactttgtagaccaggctggcctcgaactcagaaatccgcctgcctctgcctcctgagtgccgggattaaaggcgtgcaccaccacgcctggctaagttggatattttgttatataactataaccaatactaactccactgggtggatttttaattcagtcagtagtcttaagtggtctttattggcccttcattaaaatctactgttcactctaacagaggctgttggtactagtggcacttaagcaacttcctacggatatactagcagattaagggtcagggatagaaactagtctagcgttttgtatacctaccagctttatactaccttgttctgatagaaatatttcaggacatctagcacccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcc

SEQ ID NO: 14 - DNA, 23 bp, Artificial
  Oligonucleotide
gacaggacagtgcttgtttaagg

SEQ ID NO: 15 - DNA, 23 bp, Artificial
  Oligonucleotide
tgactacacaatattgctcgcac

SEQ ID NO: 16 - DNA, 1073 bp, ROSA 5′
caggccctccgagcgtggtggagccgttctgtgagacagccgggtacgagtcgtgacgctggaaggggcaagcgggtggtgggcaggaatgcggtccgccctgcagcaaccggagggggagggagaagggagcggaaaagtctccaccggacgcggccatggctcgggggggggggggcagcggaggagcgcttccggccgacgtctcgtcgctgattggcttcttttcctcccgccgtgtgtgaaaacacaaatggcgtgttttggttggcgtaaggcgcctgtcagttaacggcagccggagtgcgcagccgccggcagcctcgctctgcccactgggtggggcgggaggtaggtggggtgaggcgagctggacgtgcgggcgcggtcggcctctggcggggcgggggaggggagggagggtcagcgaaagtagctcgcgcgcgagcggccgcccaccctccccttcctctgggggagtcgttttacccgccgccggccgggcctcgtcgtctgattggctctcggggcccagaaaactggcccttgccattggctcgtgttcgtgcaagttgagtccatccgccggccagcgggggcggcgaggaggcgctcccaggttccggccctcccctcggccccgcgccgcagagtctggccgcgcgcccctgcgcaacgtggcaggaagcgcgcgctgggggcggggacgggcagtagggctgagcggctgcggggcgggtgcaagcacgtttccgacttgagttgcctcaagaggggcgtgctgagccagacctccatcgcgcactccggggagtggagggaaggagcgagggctcagttgggctgttttggaggcaggaagcacttgctctcccaaagtcgctctgagttgttatcagtaagggagctgcagtggagtaggcggggagaaggccgcacccttctccggaggggggaggggagtgttgcaatacctttctgggagttctctgctgcctcctggcttctgaggaccgccctgggcctgggagaatcccttccccctcttccctcgtgatctgcaactccagtct

SEQ ID NO: 17 - DNA, 4333 bp, ROSA 3′
tagaagatgggcgggagtcttctgggcaggcttaaaggctaacctggtgtgtgggcgttgtcctgcaggggaattgaacaggtgtaaaattggagggacaagacttcccacagattttcggttttgtcgggaagttttttaataggggcaaataaggaaaatgggaggataggtagtcatctggggttttatgcagcaaaactacaggttattattgcttgtgatccgcctcggagtattttccatcgaggtagattaaagacatgctcacccgagttttatactctcctgcttgagatccttactacagtatgaaattacagtgtcgcgagttagactatgtaagcagaattttaatcatttttaaagagcccagtacttcatatccatttctcccgctccttctgcagccttatcaaaaggtattttagaacactcattttagccccattttcatttattatactggcttatccaacccctagacagagcattggcattttccctttcctgatcttagaagtctgatgactcatgaaaccagacagattagttacatacaccacaaatcgaggctgtagctggggcctcaacactgcagttcttttataactccttagtacactttttgttgatcctttgccttgatccttaattttcagtgtctatcacctctcccgtcagtggtgttccacatttgggcctattctcagtccagggagttttacaacaatagatgtattgagaatccaacctaaagcttaactttccactcccatgaatgcctctctcctttttctccatttataaactgagctattaaccattaatggttccaggtggatgtctcctccccatattacctgatgtatcttacatattgccaggctgatattttaagacattaaaaggtatatttcattattgagccacatggtattgattactgcttactaaaattttgtcattgtacacatctgtaaaaggtggttccttttggaatgcaaagttcaggtgtttgttgtctttcctgacctaaggtcttgtgagcttgtattttttctatttaagcagtgctttctcttggactggcttgactcatggcattctacacgttattgctggtctaaatgtgattttgccaagcttcttcaggacctataattttgcttgacttgtagccaaacacaagtaaaatgattaagcaacaaatgtatttgtgaagcttggtttttaggttgttgtgttgtgtgtgcttgtgctctataataatactatccaggggctggagaggtggctcggagttcaagagcacagactgctcttccagaagtcctgagttcaattcccagcaaccacatggtggctcacaaccatctgtaatgggatctgatgccctcttctggtgtgtctgaagaccacaagtgtattcacattaaataaataaatcctccttcttcttcttttttttttttttaaagagaatactgtctccagtagaatttactgaagtaatgaaatactttgtgtttgttccaatatggtagccaataatcaaattactctttaagcactggaaatgttaccaaggaactaatttttatttgaagtgtaactgtggacagaggagccataactgcagacttgtgggatacagaagaccaatgcagactttaatgtcttttctcttacactaagcaataaagaaataaaaattgaacttctagtatcctatttgtttaaactgctagctttacttaacttttgtgcttcatctatacaaagctgaaagctaagtctgcagccattactaaacatgaaagcaagtaatgataattttggatttcaaaaatgtagggccagagtttagccagccagtggtggtgcttgcctttatgcctttaatcccagcactctggaggcagagacaggcagatctctgagtttgagcccagcctggtctacacatcaagttctatctaggatagccaggaatacacacagaaaccctgttggggaggggggctctgagatttcataaaattataattgaagcattccctaatgagccactatggatgtggctaaatccgtctacctttctgatgagatttgggtattattttttctgtctctgctgttggttgggtcttttgacactgtgggctttctttaaagcctccttcctgccatgtggtctcttgtttgctactaacttcccatggcttaaatggcatggctttttgccttctaagggcagctgctgagatttgcagcctgatttccagggtggggttgggaaatctttcaaacactaaaattgtcctttaattttttttttaaaaaatgggttatataataaacctcataaaatagttatgaggagtgaggtggactaatattaaatgagtccctcccctataaaagagctattaaggctttttgtcttatacttaactttttttttaaatgtggtatctttagaaccaagggtcttagagttttagtatacagaaactgttgcatcgcttaatcagattttctagtttcaaatccagagaatccaaattcttcacagccaaagtcaaattaagaatttctgacttttaatgttaatttgcttactgtgaatataaaaatgatagcttttcctgaggcagggtctcactatgtatctctgcctgatctgcaacaagatatgtagactaaagttctgcctgcttttgtctcctgaatactaaggttaaaatgtagtaatacttttggaacttgcaggtcagattcttttataggggacacactaagggagcttgggtgatagttggtaaaatgtgtttcaagtgatgaaaacttgaattattatcaccgcaacctactttttaaaaaaaaaagccaggcctgttagagcatgcttaagggatccctaggacttgctgagcacacaagagtagttacttggcaggctcctggtgagagcatatttcaaaaaacaaggcagacaaccaagaaactacagttaaggttacctgtctttaaaccatctgcatatacacagggatattaaaatattccaaataatatttcattcaagttttcccccatcaaattgggacatggatttctccggtgaataggcagagttggaaactaaacaaatgttggttttgtgatttgtgaaattgttttcaagtgatagttaaagcccatgagatacagaacaaagctgctatttcgaggtctcttggtttatactcagaagcacttctttgggtttccctgcactatcctgatcatgtgctaggcctaccttaggctgattgttgttcaaataaacttaagtttcctgtcaggtgatgtcatatgatttcatatatcaaggcaaaacatgttatatatgttaaacatttgtacttaatgtgaaagttaggtctttgtgggtttgatttttaattttcaaaacctgagctaaataagtcatttttacatgtcttacatttggtggaattgtataattgtggtttgcaggcaagactctctgacctagtaaccctacctatagagcactttgctgggtcacaagtctaggagtcaagcatttcaccttgaagttgagacgttttgttagtgtatactagtttatatgttggaggacatgtttatccagaagatattcaggactatttttgactgggctaaggaattgattctgattagcactgttagtgagcattgagtggcctttaggcttgaattggagtcacttgtatatctcaaataatgctggccttttttaaaaagcccttgttctttatcaccctgttttctacataatttttgttcaaagaaatacttgtttggatctccttttgacaacaatagcatgttttcaagccatattttttttcctttttttttttttttttggtttttcgagacagggtttctctgtatagccctggctgtcctggaactcactttgtagaccaggctggcctcgaactcagaaatccgcctgcctctgcctcctgagtgccgggattaaaggcgtgcaccaccacgcctggctaagttggatattttgttatataactataaccaatactaactccactgggtggatttttaattcagtcagtagtcttaagtggtctttattggcccttcattaaaatctactgttcactctaacagaggctgttggtactagtggcacttaagcaacttcctacggatatactagcagattaagggtcagggatagaaactagtctagcgttttgtatacctaccagctttatactaccttgttctgatagaaatatttcaggacatc

SEQ ID NO: 18 - DNA, 10491 bp, pROSA 12
tttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctgtcgagatctagatatcgatggccatagagttacgctagggataacagggtaatatagccgcggcaggccctccgagcgtggtggagccgttctgtgagacagccgggtacgagtcgtgacgctggaaggggcaagcgggtggtgggcaggaatgcggtccgccctgcagcaaccggagggggagggagaagggagcggaaaagtctccaccggacgcggccatggctcgggggggggggggcagcggaggagcgcttccggccgacgtctcgtcgctgattggcttcttttcctcccgccgtgtgtgaaaacacaaatggcgtgttttggttggcgtaaggcgcctgtcagttaacggcagccggagtgcgcagccgccggcagcctcgctctgcccactgggtggggcgggaggtaggtggggtgaggcgagctggacgtgcgggcgcggtcggcctctggcggggcgggggaggggagggagggtcagcgaaagtagctcgcgcgcgagcggccgcccaccctccccttcctctgggggagtcgttttacccgccgccggccgggcctcgtcgtctgattggctctcggggcccagaaaactggcccttgccattggctcgtgttcgtgcaagttgagtccatccgccggccagcgggggcggcgaggaggcgctcccaggttccggccctcccctcggccccgcgccgcagagtctggccgcgcgcccctgcgcaacgtggcaggaagcgcgcgctgggggcggggacgggcagtagggctgagcggctgcggggcgggtgcaagcacgtttccgacttgagttgcctcaagaggggcgtgctgagccagacctccatcgcgcactccggggagtggagggaaggagcgagggctcagttgggctgttttggaggcaggaagcacttgctctcccaaagtcgctctgagttgttatcagtaagggagctgcagtggagtaggcggggagaaggccgcacccttctccggaggggggaggggagtgttgcaatacctttctgggagttctctgctgcctcctggcttctgaggaccgccctgggcctgggagaatcccttccccctcttccctcgtgatctgcaactccagtctttctaggtaaccgatatccctgcaggggtgacctgcacgtctagggcgcagtagtccagggtttccttgatgatgtcatacttatcctgtcccttttttttccacagctcgcggttgaggacaaactcttcgcggtctttccagtactcctgcaggtgactgactgagtcgacttaattaaggccatagcggccatttaaatcggccggcctaggcgcgccggtaaccgaagttcctatactttctagagaataggaacttcggaataggaacttcttaggtcaattctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacctgcagccaatatgggatcggccattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaggggatcgatccgctgtaagtctgcagaaattgatgatctattaaacaataaagatgtccactaaaatggaagtttttcctgtcatactttgttaagaagggtgagaacagagtacctacattttgaatggaaggattggagctacgggggtgggggtggggtgggattagataaatgcctgctctttactgaaggctctttactattgctttatgataatgtttcatagttggatatcataatttaaacaagcaaaaccaaattaagggccagctcattcctcccactcatgatctatagatctatagatctctcgtgggatcattgtttttctcttgattcccactttgtggttctaagtactgtggtttccaaatgtgtcagtttcatagcctgaagaacgagatcagcagcctctgttccacatacacttcattctcagtattgttttgccaagttctaattccatcagaagctgactctagatcccgcgccgaagttcctatactttctagagaataggaacttcggaataggaacttcaagcttaagcgctagaagatgggcgggagtcttctgggcaggcttaaaggctaacctggtgtgtgggcgttgtcctgcaggggaattgaacaggtgtaaaattggagggacaagacttcccacagattttcggttttgtcgggaagttttttaataggggcaaataaggaaaatgggaggataggtagtcatctggggttttatgcagcaaaactacaggttattattgcttgtgatccgcctcggagtattttccatcgaggtagattaaagacatgctcacccgagttttatactctcctgcttgagatccttactacagtatgaaattacagtgtcgcgagttagactatgtaagcagaattttaatcatttttaaagagcccagtacttcatatccatttctcccgctccttctgcagccttatcaaaaggtattttagaacactcattttagccccattttcatttattatactggcttatccaacccctagacagagcattggcattttccctttcctgatcttagaagtctgatgactcatgaaaccagacagattagttacatacaccacaaatcgaggctgtagctggggcctcaacactgcagttcttttataactccttagtacactttttgttgatcctttgccttgatccttaattttcagtgtctatcacctctcccgtcagtggtgttccacatttgggcctattctcagtccagggagttttacaacaatagatgtattgagaatccaacctaaagcttaactttccactcccatgaatgcctctctcctttttctccatttataaactgagctattaaccattaatggttccaggtggatgtctcctccccatattacctgatgtatcttacatattgccaggctgatattttaagacattaaaaggtatatttcattattgagccacatggtattgattactgcttactaaaattttgtcattgtacacatctgtaaaaggtggttccttttggaatgcaaagttcaggtgtttgttgtctttcctgacctaaggtcttgtgagcttgtattttttctatttaagcagtgctttctcttggactggcttgactcatggcattctacacgttattgctggtctaaatgtgattttgccaagcttcttcaggacctataattttgcttgacttgtagccaaacacaagtaaaatgattaagcaacaaatgtatttgtgaagcttggtttttaggttgttgtgttgtgtgtgcttgtgctctataataatactatccaggggctggagaggtggctcggagttcaagagcacagactgctcttccagaagtcctgagttcaattcccagcaaccacatggtggctcacaaccatctgtaatgggatctgatgccctcttctggtgtgtctgaagaccacaagtgtattcacattaaataaataaatcctccttcttcttcttttttttttttttaaagagaatactgtctccagtagaatttactgaagtaatgaaatactttgtgtttgttccaatatggtagccaataatcaaattactctttaagcactggaaatgttaccaaggaactaatttttatttgaagtgtaactgtggacagaggagccataactgcagacttgtgggatacagaagaccaatgcagactttaatgtcttttctcttacactaagcaataaagaaataaaaattgaacttctagtatcctatttgtttaaactgctagctttacttaacttttgtgcttcatctatacaaagctgaaagctaagtctgcagccattactaaacatgaaagcaagtaatgataattttggatttcaaaaatgtagggccagagtttagccagccagtggtggtgcttgcctttatgcctttaatcccagcactctggaggcagagacaggcagatctctgagtttgagcccagcctggtctacacatcaagttctatctaggatagccaggaatacacacagaaaccctgttggggaggggggctctgagatttcataaaattataattgaagcattccctaatgagccactatggatgtggctaaatccgtctacctttctgatgagatttgggtattattttttctgtctctgctgttggttgggtcttttgacactgtgggctttctttaaagcctccttcctgccatgtggtctcttgtttgctactaacttcccatggcttaaatggcatggctttttgccttctaagggcagctgctgagatttgcagcctgatttccagggtggggttgggaaatctttcaaacactaaaattgtcctttaattttttttttaaaaaatgggttatataataaacctcataaaatagttatgaggagtgaggtggactaatattaaatgagtccctcccctataaaagagctattaaggctttttgtcttatacttaactttttttttaaatgtggtatctttagaaccaagggtcttagagttttagtatacagaaactgttgcatcgcttaatcagattttctagtttcaaatccagagaatccaaattcttcacagccaaagtcaaattaagaatttctgacttttaatgttaatttgcttactgtgaatataaaaatgatagcttttcctgaggcagggtctcactatgtatctctgcctgatctgcaacaagatatgtagactaaagttctgcctgcttttgtctcctgaatactaaggttaaaatgtagtaatacttttggaacttgcaggtcagattcttttataggggacacactaagggagcttgggtgatagttggtaaaatgtgtttcaagtgatgaaaacttgaattattatcaccgcaacctactttttaaaaaaaaaagccaggcctgttagagcatgcttaagggatccctaggacttgctgagcacacaagagtagttacttggcaggctcctggtgagagcatatttcaaaaaacaaggcagacaaccaagaaactacagttaaggttacctgtctttaaaccatctgcatatacacagggatattaaaatattccaaataatatttcattcaagttttcccccatcaaattgggacatggatttctccggtgaataggcagagttggaaactaaacaaatgttggttttgtgatttgtgaaattgttttcaagtgatagttaaagcccatgagatacagaacaaagctgctatttcgaggtctcttggtttatactcagaagcacttctttgggtttccctgcactatcctgatcatgtgctaggcctaccttaggctgattgttgttcaaataaacttaagtttcctgtcaggtgatgtcatatgatttcatatatcaaggcaaaacatgttatatatgttaaacatttgtacttaatgtgaaagttaggtctttgtgggtttgatttttaattttcaaaacctgagctaaataagtcatttttacatgtcttacatttggtggaattgtataattgtggtttgcaggcaagactctctgacctagtaaccctacctatagagcactttgctgggtcacaagtctaggagtcaagcatttcaccttgaagttgagacgttttgttagtgtatactagtttatatgttggaggacatgtttatccagaagatattcaggactatttttgactgggctaaggaattgattctgattagcactgttagtgagcattgagtggcctttaggcttgaattggagtcacttgtatatctcaaataatgctggccttttttaaaaagcccttgttctttatcaccctgttttctacataatttttgttcaaagaaatacttgtttggatctccttttgacaacaatagcatgttttcaagccatattttttttcctttttttttttttttttggtttttcgagacagggtttctctgtatagccctggctgtcctggaactcactttgtagaccaggctggcctcgaactcagaaatccgcctgcctctgcctcctgagtgccgggattaaaggcgtgcaccaccacgcctggctaagttggatattttgttatataactataaccaatactaactccactgggtggatttttaattcagtcagtagtcttaagtggtctttattggcccttcattaaaatctactgttcactctaacagaggctgttggtactagtggcacttaagcaacttcctacggatatactagcagattaagggtcagggatagaaactagtctagcgttttgtatacctaccagctttatactaccttgttctgatagaaatatttcaggacatctagcacccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcc

SEQ ID NO: 19 - DNA, 448 bp, ROSA5.1
aaggatactggggcatacgccacagggagtccaagaatgtgaggtgggggtggcgaaggtaatgtctttggtgtgggaaaagcagcagccatctgagataggaactggaaaaccagaggagaggcgttcaggaagattatggaggggaggactgggcccccacgagcgaccagagttgtcacaaggccgcaagaacaggggaggtggggggctcagggacagaaaaaaaagtatgtgtattttgagagcagggttgggaggcctctcctgaaaagggtataaacgtggagtaggcaatacccaggcaaaaaggggagaccagagtagggggaggggaagagtcctgacccagggaagacattaaaaaggtagtggggtcgactagatgaaggagagcctttctctctgggcaagagcggtgcaatggtgtgtaaaggtagctgagaag

SEQ ID NO: 20 - DNA, 11784 bp, C31 substrate reporter
ggcaggccctccgagcgtggtggagccgttctgtgagacagccgggtacgagtcgtgacgctggaaggggcaagcgggtggtgggcaggaatgcggtccgccctgcagcaaccggagggggagggagaagggagcggaaaagtctccaccggacgcggccatggctcgggggggggggggcagcggaggagcgcttccggccgacgtctcgtcgctgattggcttcttttcctcccgccgtgtgtgaaaacacaaatggcgtgttttggttggcgtaaggcgcctgtcagttaacggcagccggagtgcgcagccgccggcagcctcgctctgcccactgggtggggcgggaggtaggtggggtgaggcgagctggacgtgcgggcgcggtcggcctctggcggggcgggggaggggagggagggtcagcgaaagtagctcgcgcgcgagcggccgcccaccctccccttcctctgggggagtcgttttacccgccgccggccgggcctcgtcgtctgattggctctcggggcccagaaaactggcccttgccattggctcgtgttcgtgcaagttgagtccatccgccggccagcgggggcggcgaggaggcgctcccaggttccggccctcccctcggccccgcgccgcagagtctggccgcgcgcccctgcgcaacgtggcaggaagcgcgcgctgggggcggggacgggcagtagggctgagcggctgcggggcgggtgcaagcacgtttccgacttgagttgcctcaagaggggcgtgctgagccagacctccatcgcgcactccggggagtggagggaaggagcgagggctcagttgggctgttttggaggcaggaagcacttgctctcccaaagtcgctctgagttgttatcagtaagggagctgcagtggagtaggcggggagaaggccgcacccttctccggaggggggaggggagtgttgcaatacctttctgggagttctctgctgcctcctggcttctgaggaccgccctgggcctgggagaatcccttccccctcttccctcgtgatctgcaactccagtctttcgcctaggtaaccgatatccctgcaggggtgacctgcacgtctagggcgcagtagtccagggtttccttgatgatgtcatacttatcctgtcccttttttttccacagctcgcggttgaggacaaactcttcgcggtctttccagtactcctgcaggtgactgactgagtcgacgacactgcagagacctacttcactaacaaccggtacagttcgtggaccagatgggtgaggtggagtacgcgcccggggagcccaagggcacgccctggcacccgcaccgcggcttcgagaccgtcacgaataacttcgtatagcatacattatacgaagttataagctcgatgaattctaccgggtaggggaggcgcttttcccaaggcagtctggagcatgcgctttagcagccccgctggcacttggcgctacacaagtggcctctggcctcgcacacattccacatccaccggtagcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcgaaggtcctcccgaggcccggcattctcgcacgcttcaaaagcgcacgtctgccgcgctgttctcctcttcctcatctccgggcctttcgacgatccagccgccaccatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagtcgatgcagaaattgatgatctattaaacaataaagatgtccactaaaatggaagtttttcctgtcatactttgttaagaagggtgagaacagagtacctacattttgaatggaaggattggagctacgggggtgggggtggggtgggattagataaatgcctgctctttactgaaggctctttactattgctttatgataatgtttcatagttggatatcataatttaaacaagcaaaaccaaattaagggccagctcattcctcccactcatgatctatagatctatagatctctcgtgggatcattgtttttctcttgattcccactttgtggttctaagtactgtggtttccaaatgtgtcagtttcatagcctgaagaacgagatcagcagcctctgttccacatacacttcattctcagtattgttttgccaagttctaattccatcagaagcttcagctgctcgactagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatccgtgtcatgtcggcgaccctacgcccccaactgagagaactcaaaggttaccccagttggggcactactcccgaaaaccgcttctggatccataacttcgtatagcatacattatacgaagttataccgggccaccatggtcgcgagtagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtaacctatcccattacggtcaatccgccgtttgttcccacggagaatccgacgggttgttactcgctcacatttaatgttgatgaaagctggctacaggaaggccagacgcgaattatttttgatggcgttaactcggcgtttcatctgtggtgcaacgggcgctgggtcggttacggccaggacagtcgtttgccgtctgaatttgacctgagcgcatttttacgcgccggagaaaaccgcctcgcggtgatggtgctgcgttggagtgacggcagttatctggaagatcaggatatgtggcggatgagcggcattttccgtgacgtctcgttgctgcataaaccgactacacaaatcagcgatttccatgttgccactcgctttaatgatgatttcagccgcgctgtactggaggctgaagttcagatgtgcggcgagttgcgtgactacctacgggtaacagtttctttatggcagggtgaaacgcaggtcgccagcggcaccgcgcctttcggcggtgaaattatcgatgagcgtggtggttatgccgatcgcgtcacactacgtctgaacgtcgaaaacccgaaactgtggagcgccgaaatcccgaatctctatcgtgcggtggttgaactgcacaccgccgacggcacgctgattgaagcagaagcctgcgatgtcggtttccgcgaggtgcggattgaaaatggtctgctgctgctgaacggcaagccgttgctgattcgaggcgttaaccgtcacgagcatcatcctctgcatggtcaggtcatggatgagcagacgatggtgcaggatatcctgctgatgaagcagaacaactttaacgccgtgcgctgttcgcattatccgaaccatccgctgtggtacacgctgtgcgaccgctacggcctgtatgtggtggatgaagccaatattgaaacccacggcatggtgccaatgaatcgtctgaccgatgatccgcgctggctaccggcgatgagcgaacgcgtaacgcgaatggtgcagcgcgatcgtaatcacccgagtgtgatcatctggtcgctggggaatgaatcaggccacggcgctaatcacgacgcgctgtatcgctggatcaaatctgtcgatccttcccgcccggtgcagtatgaaggcggcggagccgacaccacggccaccgatattatttgcccgatgtacgcgcgcgtggatgaagaccagcccttcccggctgtgccgaaatggtccatcaaaaaatggctttcgctacctggagagacgcgcccgctgatcctttgcgaatacgcccacgcgatgggtaacagtcttggcggtttcgctaaatactggcaggcgtttcgtcagtatccccgtttacagggcggcttcgtctgggactgggtggatcagtcgctgattaaatatgatgaaaacggcaacccgtggtcggcttacggcggtgattttggcgatacgccgaacgatcgccagttctgtatgaacggtctggtctttgccgaccgcacgccgcatccagcgctgacggaagcaaaacaccagcagcagtttttccagttccgtttatccgggcaaaccatcgaagtgaccagcgaatacctgttccgtcatagcgataacgagctcctgcactggatggtggcgctggatggtaagccgctggcaagcggtgaagtgcctctggatgtcgctccacaaggtaaacagttgattgaactgcctgaactaccgcagccggagagcgccgggcaactctggctcacagtacgcgtagtgcaaccgaacgcgaccgcatggtcagaagccgggcacatcagcgcctggcagcagtggcgtctggcggaaaacctcagtgtgacgctccccgccgcgtcccacgccatcccgcatctgaccaccagcgaaatggatttttgcatcgagctgggtaataagcgttggcaatttaaccgccagtcaggctttctttcacagatgtggattggcgataaaaaacaactgctgacgccgctgcgcgatcagttcacccgtgcaccgctggataacgacattggcgtaagtgaagcgacccgcattgaccctaacgcctgggtcgaacgctggaaggcggcgggccattaccaggccgaagcagcgttgttgcagtgcacggcagatacacttgctgatgcggtgctgattacgaccgctcacgcgtggcagcatcaggggaaaaccttatttatcagccggaaaacctaccggattgatggtagtggtcaaatggcgattaccgttgatgttgaagtggcgagcgatacaccgcatccggcgcggattggcctgaactgccagctggcgcaggtagcagagcgggtaaactggctcggattagggccgcaagaaaactatcccgaccgccttactgccgcctgttttgaccgctgggatctgccattgtcagacatgtataccccgtacgtcttcccgagcgaaaacggtctgcgctgcgggacgcgcgaattgaattatggcccacaccagtggcgcggcgacttccagttcaacatcagccgctacagtcaacagcaactgatggaaaccagccatcgccatctgctgcacgcggaagaaggcacatggctgaatatcgacggtttccatatggggattggtggcgacgactcctggagcccgtcagtatcggcggaattccagctgagcgccggtcgctaccattaccagttggtctggtgtcaaaaataataataaccgggcaggggggatctttgtgaaggaaccttacttctgtggtgtgacataattggacaaactacctacagagatttaaagctctaaggtaaatataaaatttttaagtgtataatgtgttaaactactgattctaattgtttgtgtattttagattccaacctatggaactgatgaatgggagcagtggtggaatgccagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatctgcggccaaatcggccggcctaggcgcgccggtaaccgaagttcctatactttctagagaataggaacttcggaataggaacttcaagcttaagcgctagcctagaagatgggcgggagtcttctgggcaggcttaaaggctaacctggtgtgtgggcgttgtcctgcaggggaattgaacaggtgtaaaattggagggacaagacttcccacagattttcggttttgtcgggaagttttttaataggggcaaataaggaaaatgggaggataggtagtcatctggggttttatgcagcaaaactacaggttattattgcttgtgatccgcctcggagtattttccatcgaggtagattaaagacatgctcacccgagttttatactctcctgcttgagatccttactacagtatgaaattacagtgtcgcgagttagactatgtaagcagaattttaatcatttttaaagagcccagtacttcatatccatttctcccgctccttctgcagccttatcaaaaggtattttagaacactcattttagccccattttcatttattatactggcttatccaacccctagacagagcattggcattttccctttcctgatcttagaagtctgatgactcatgaaaccagacagattagttacatacaccacaaatcgaggctgtagctggggcctcaacactgcagttcttttataactccttagtacactttttgttgatcctttgccttgatccttaattttcagtgtctatcacctctcccgtcagtggtgttccacatttgggcctattctcagtccagggagttttacaacaatagatgtattgagaatccaacctaaagcttaactttccactcccatgaatgcctctctcctttttctccatttataaactgagctattaaccattaatggttccaggtggatgtctcctccccatattacctgatgtatcttacatattgccaggctgatattttaagacattaaaaggtatatttcattattgagccacatggtattgattactgcttactaaaattttgtcattgtacacatctgtaaaaggtggttccttttggaatgcaaagttcaggtgtttgttgtctttcctgacctaaggtcttgtgagcttgtattttttctatttaagcagtgctttctcttggactggcttgactcatggcattctacacgttattgctggtctaaatgtgattttgccaagcttcttcaggacctataattttgcttgacttgtagccaaacacaagtaaaatgattaagcaacaaatgtatttgtgaagcttggtttttaggttgttgtgttgtgtgtgcttgtgctctataataatactatccaggggctggagaggtggctcggagttcaagagcacagactgctcttccagaagtcctgagttcaattcccagcaaccacatggtggctcacaaccatctgtaatgggatctgatgccctcttctggtgtgtctgaagaccacaagtgtattcacattaaataaataaatcctccttcttcttcttttttttttttttaaagagaatactgtctccagtagaatttactgaagtaatgaaatactttgtgtttgttccaatatggtagccaataatcaaattactctttaagcactggaaatgttaccaaggaactaatttttatttgaagtgtaactgtggacagaggagccataactgcagacttgtgggatacagaagaccaatgcagactttaatgtcttttctcttacactaagcaataaagaaataaaaattgaacttctagtatcctatttgtttaaactgctagctttacttaacttttgtgcttcatctatacaaagctgaaagctaagtctgcagccattactaaacatgaaagcaagtaatgataattttggatttcaaaaatgtagggccagagtttagccagccagtggtggtgcttgcctttatgcctttaatcccagcactctggaggcagagacaggcagatctctgagtttgagcccagcctggtctacacatcaagttctatctaggatagccaggaatacacacagaaaccctgttggggaggggggctctgagatttcataaaattataattgaagcattccctaatgagccactatggatgtggctaaatccgtctacctttctgatgagatttgggtattattttttctgtctctgctgttggttgggtcttttgacactgtgggctttctttaaagcctccttcctgccatgtggtctcttgtttgctactaacttcccatggcttaaatggcatggctttttgccttctaagggcagctgctgagatttgcagcctgatttccagggtggggttgggaaatctttcaaacactaaaattgtcctttaattttttttttaaaaaatgggttatataataaacctcataaaatagttatgaggagtgaggtggactaatattaaatgagtccctcccctataaaagagctattaaggctttttgtcttatacttaactttttttttaaatgtggtatctttagaaccaagggtcttagagttttagtatacagaaactgttgcatcgcttaatcagattttctagtttcaaatccagagaatccaaattcttcacagccaaagtcaaattaagaatttctgacttttaatgttaatttgcttactgtgaatataaaaatgatagcttttcctgaggcagggtctcactatgtatctctgcctgatctgcaacaagatatgtagactaaagttctgcctgcttttgtctcctgaatactaaggttaaaatgtagtaatacttttggaacttgcaggtcagattcttttataggggacacactaagggagcttgggtgatagttggtaaaatgtgtttcaagtgatgaaaacttgaattattatcaccgcaacctactttttaaaaaaaaaagccaggcctgttagagcatgcttaagggatccctaggacttgctgagcacacaagagtagttacttggcaggctcctggtgagagcatatttcaaaaaacaaggcagacaaccaagaaactacagttaaggttacctgtctttaaaccatctgcatatacacagggatattaaaatattccaaataatatttcattcaagttttcccccatcaaattgggacatggatttctccggtgaataggcagagttggaaactaaacaaatgttggttttgtgatttgtgaaattgttttcaagtgatagttaaagcccatgagatacagaacaaagctgctatttcgaggtctcttggtttatactcagaagcacttctttgggtttccctgcactatcctgatcatgtgctaggcctaccttaggctgattgttgttcaaataaacttaagtttcctgtcaggtgatgtcatatgatttcatatatcaaggcaaaacatgttatatatgttaaacatttgtacttaatgtgaaagttaggtctttgtgggtttgatttttaattttcaaaacctgagctaaataagtcatttttacatgtcttacatttggtggaattgtataattgtggtttgcaggcaagactctctgacctagtaaccctacctatagagcactttgctgggtcacaagtctaggagtcaagcatttcaccttgaagttgagacgttttgttagtgtatactagtttatatgttggaggacatgtttatccagaagatattcaggactatttttgactgggctaaggaattgattctgattagcactgttagtgagcattgagtggcctttaggcttgaattggagtcacttgtatatctcaaataatgctggccttttttaaaaagcccttgttctttatcaccctgttttctacataatttttgttcaaagaaatacttgtttggatctccttttgacaacaatagcatgttttcaagccatattttttttcctttttttttttttttttggtttttcgagacagggtttctctgtatagccctggctgtcctggaactcactttgtagaccaggctggcctcgaactcagaaatccgcctgcctctgcctcctgagtgccgggattaaaggcgtgcaccaccacgcctggctaagttggatattttgttatataactataaccaatactaactccactgggtggatttttaattcagtcagtagtcttaagtggtctttattggcccttcattaaaatctactgttcactctaacagaggctgttggtactagtggcacttaagcaacttcctacggatatactagcagattaagggtcagggatagaaactagtctagcgttttgtatacctaccagctttatactaccttgttctgatagaaatatttcaggacat

SEQ ID NO: 21 - DNA, 20 bp, Artificial
  oligonucleotide
gcgtgagatcaagacgcaca

SEQ ID NO: 22 - DNA, 21 bp, Artificial
  Oligonucleotide
gcagcggtaagagtccttgat

SEQ ID NO: 23 - DNA, 19 bp, Artificial
  Oligonucleotide
gtgaacgtggtgatcaaca

SEQ ID NO: 24 - DNA, 19 bp, Artificial
  Oligonucleotide
gcagtacagcttgtccatg

SEQ ID NO: 25 - DNA, 20 bp, Artificial
  Oligonucleotide
atcctctgcatggtcaggtc

SEQ ID NO: 26 - DNA, 18 bp, Artificial
  Oligonucleotide
cgtggcctgattcattcc